(19) United States
(12) Patent Application Publication
(10) Pub. No.: US 2026/0273306 A1
(43) Pub. Date: Sep. 17, 2026

(54) PHOTOTHERAPY APPARATUS FOR TREATING ALZHEIMER'S DISEASE AND ASSOCIATED CONDITIONS

(71) Applicant: DANYANG HUICHUANG MEDICAL EQUIPMENT CO., LTD., Zhenjiang (CN)

(72) Inventors: Daifa WANG, Zhenjiang (CN); Genmiao GUO, Zhenjiang (CN); Xiaojing ZHAO, Zhenjiang (CN)

(73) Assignee: DANYANG HUICHUANG MEDICAL EQUIPMENT CO., LTD., Zhenjiang (CN)

(21) Appl. No.: 19/678,012

(22) Filed: May 14, 2026

Related U.S. Application Data
(63) Continuation of application No. PCT/CN2024/125348, filed on Oct. 16, 2024.

(30) Foreign Application Priority Data
Nov. 15, 2023 (CN) 202323093294.0

(51) Int. Cl. A61N 5/06

(52) U.S. Cl. CPC A61N 5/0622; A61N 2005/0626; A61N 2005/0647; A61N 2005/0659

(57) ABSTRACT

A phototherapy apparatus for treating Alzheimer's disease and associated conditions includes a support mechanism forming an accommodation space and an array of near-infrared irradiation units. The near-infrared light delivered to the subject's head satisfies a coordinated irradiation condition involving an irradiated surface area ratio and a delivered radiant power level. This ratio, ranging from 30% to 65% or more, is determined relative to a reference calvarial region. The surface area of the reference calvarial region is the external surface area of the subject's head within a total boundary line extending from a Glabella point along supraorbital ridges, passes through preauricular points on both sides, curves posteriorly around the head to pass between the inion and the electrode positions O1, OZ, and O2 of the international 10-10 standard system, and then converges, and the irradiated surface area ratio is in the range of 30% to 65% or more.

### CROSS REFERENCE TO RELATED APPLICATIONS

This application is a U.S. bypass continuation of International Application No. PCT/CN2024/125348, filed on Oct. 16, 2024, which claims priority to Chinese Application No. 202323093294.0, filed on Nov. 15, 2023. The entire contents of each of the above-referenced applications are expressly incorporated herein by reference.

### TECHNICAL FIELD

The present application belongs to the technical field of phototherapy apparatuses for cerebral and cognitive diseases, and in particular, relates to a phototherapy apparatus for treating Alzheimer's disease and associated conditions.

### BACKGROUND

Alzheimer's disease (AD) is a chronic and progressive neurodegenerative disease that mainly affects the elderly, especially those over 60 years old. The disease is characterized by memory loss, impairment of social and occupational functions, decline of executive function, speech and motor deficits, personality changes, as well as behavioral and psychological disorders. The progressive course of the disease may last for 8 to 10 years, and currently, it cannot be cured completely, bringing a heavy burden to families and society.

At present, the pathogenesis of AD is not fully understood, but the relatively recognized mechanisms are as follows: abnormal processing of amyloid precursor protein (APP) produces β-amyloid (Aβ), which aggregates to form amyloid plaques; abnormal phosphorylation of tau protein forms neurofibrillary tangles (NFTs), and the accumulation of such tangles in the brain leads to neuronal damage and dysfunction. Furthermore, intracellular NFTs and extracellular Aβ deposition may form senile plaques, resulting in an increase of oxidative stress, an increase of neuroinflammation, and mitochondrial dysfunction or the like, which in turn causes neuronal dysfunction and synaptic loss, and ultimately leads to neuronal death.

Long-term clinical practice has demonstrated that pharmacotherapy, such as cholinesterase inhibitors, memantine, and the recently emerged lecanemab (a humanized monoclonal antibody that can reduce β-amyloid deposition in the brain), combined with conventional treatment, can usually only improve mild cognitive impairment (MCI) or mild AD, and also has the problem of frequent adverse gastrointestinal reactions.

In recent years, Photobiomodulation (PBM) has been introduced for the treatment of AD. PBM involves irradiating red or near-infrared light with a wavelength range of 600 to 1100 nm to the head, which penetrates the scalp and skull to act on brain tissues, and treats AD non-invasively through various action mechanisms, such as increasing clearance for Aβ, reducing abnormal aggregation of tau protein, improving metabolism and mitochondrial function, increasing cerebral blood flow, and enhancing anti-oxidative stress and anti-inflammatory capabilities.

However, the definitions of irradiation parameters of various existing photobiomodulation apparatuses (also referred to as phototherapy apparatuses herein) are ambiguous, inconsistent, and significantly different. For example, although the irradiance (in mW/cm²) of near-infrared light is mentioned as an irradiation parameter, the position where the irradiance is applied is sometimes the immediate emergent surface of the LED (see Farzad Salehpour et al., Rapid Reversal of Cognitive Decline, Olfactory Dysfunction, and Quality of Life Using Multi-Modality Photobiomodulation Therapy: Case Report, Photobiomodul Photomed Laser Surg. 2019 March; 37(3):159-167), sometimes a few centimeters inward from the inner wall of the headgear (see Liang Chen et al., A Pilot Study of Near-Infrared Light Treatment for Alzheimer's Disease, Journal of Alzheimer's Disease 91 (2023) 191-201), and sometimes the depth under the dura mater (see U.S. Pat. No. 8,308,784B2). For the immediate emergent surface of the LED alone, even if the irradiances at that surface are the same for photobiomodulation apparatuses with different structures, the actual dose applied to the intracranial tissues will be significantly different. As shown in FIG. 1(a), the LED irradiation panels are provided inside the housing of the headgear, with a predetermined distance from the subject's head, and there is a spacing that causes light attenuation on this distance. As shown in FIG. 2(a), several LED irradiation panels are integrated into one irradiation unit, and the irradiation unit is placed in close contact with the subject's head. As shown in FIG. 2(b), the LED irradiation panels are suspended in air at a relatively large distance around the subject's head. Obviously, among these three phototherapy apparatuses, even if the immediate emergent surface of each LED has the same irradiance, the dose penetrating the scalp and skull to act on the brain tissues varies greatly.

In addition, irradiation parameters defined at intracorporal positions are not practical for the irradiation control of phototherapy apparatuses. For example, for the irradiance at a depth several centimeters below the dura mater as mentioned above, it is impossible to invasively detect the irradiance at a depth within the brain (i.e., at a depth several centimeters below the dura mater) every time the phototherapy apparatus is operated.

At present, manufacturers and researchers of near-infrared phototherapy apparatuses usually solely measure and list simply the irradiances at different positions as described above, and the irradiances used vary widely. For example, U.S. Pat. No. 9,993,659B2 recites that an irradiance at the light-emitting surface is about 1400 mW/cm² to about 4200 mW/cm², while Farzad Salehpour et al. Rapid Reversal of Cognitive Decline, Olfactory Dysfunction, and Quality of Life Using Multi-Modality Photobiomodulation Therapy: Case Report, Photobiomodul Photomed Laser Surg.2019 March; 37(3):159-167, recites that an irradiance at the light-emitting surface can be 31 mW/cm². With a difference of 40 to 140 times between the two, both of them report a therapeutic effect on AD. It is thus confusing what level of irradiance should be used at which position to achieve a good therapeutic effect on AD. Does solely limiting the irradiance at a certain position necessarily bring a good therapeutic effect on AD? Although manufacturers and researchers of near-infrared phototherapy apparatuses will list the power (in W) at various positions (such as the light-emitting surface of the LED), the relationship between the power and the therapeutic effect on AD in subjects is unclear. Specifically, the power they use is sometimes the luminous power of the irradiation panels and sometimes the luminous power at the brain tissue; it is unclear whether the duty cycle has been accounted for (i.e., whether it is the time-averaged luminous power or the peak luminous power), and the orders of magnitude vary significantly. Furthermore, U.S. Pat. No. 8,308,784B2 definitely records that “for a selected wavelength, the power density (light intensity or power per unit area, W/cm²) or energy density (energy per unit area, in J/cm², or power density multiplied by the exposure time) of the light energy delivered to the tissue is an important factor in determining the relative efficacy of the phototherapy, and efficacy is not directly associated with the total power or total energy delivered to the tissue” (see paragraph of its specification). Is it true that the therapeutic efficacy of near-infrared phototherapy apparatuses on AD subjects is not directly associated with the total power or total energy delivered to the tissue?

In summary, the prior art has inconsistent and even contradictory statements on the dose required for near-infrared phototherapy apparatuses, which hinders the popularization and development of near-infrared phototherapy apparatuses and even near-infrared phototherapy methods.

### SUMMARY

The present application is proposed to solve the above-mentioned problems existing in the prior art. The present application aims to provide a phototherapy apparatus for treating Alzheimer's disease and associated conditions. The phototherapy apparatus may adopt various structures, nor is it limited as to whether a near-infrared irradiation unit is an ordinary LED or a low-energy laser diode, and a robust, sustained, and significant therapeutic effect on AD and associated conditions in the treatment subjects can be achieved as long as the unified coordinated irradiation condition is satisfied.

According to a first aspect, the present application provides a phototherapy apparatus for treating Alzheimer's disease and associated conditions, which includes a support mechanism and an array of near-infrared irradiation units. The support mechanism is configured to form an accommodation space for a subject's head and to support the array of near-infrared irradiation units. The array of near-infrared irradiation units is configured to emit near-infrared light into the accommodation space. Wherein, when the subject's head is positioned within the accommodation space, the near-infrared light irradiation delivered to the subject's head satisfies a coordinated irradiation condition of an irradiated surface area ratio and a delivered radiant power level. Wherein, the irradiated surface area ratio is a ratio of an irradiated surface area of the subject's head to a surface area of a reference calvarial region of the subject's head, and the surface area of the reference calvarial region is the external surface area of a surface of the subject's head within a total boundary line. Further, the total boundary line extends from a Glabella point of the subject's head along supraorbital ridges, passes through preauricular points on both sides, curves posteriorly around the head to pass between the inion and the electrode positions O1, OZ, and O2 of the international 10-10 standard system, and then converges. The irradiated surface area ratio is in the range of 30% (which may be referred to as locally focused stimulation protocol) to 65% or more (which may be referred to as multi-region balanced stimulation protocol).

In the present application, the expression “irradiation delivered to the subject's head” refers to that the irradiation is delivered to (and thus lands on) the outer thin-layer irradiated surface that is in contact with the hair (or the scalp where there is no hair) on the subject's head. The energy irradiated (i.e., the irradiation delivered) to this outer thin-layer irradiated surface means that the energy is delivered to (i.e., is received by) the head, which includes the hair, scalp, skull, and brain tissues. Furthermore, after the delivered energy is absorbed by the hair and attenuated by the scalp and skull, the remaining energy can arrive at (acting on) the cerebral cortex and even deeper parts of the brain tissue, and the remaining energy is associated with the attenuation occurring along the transmission path.

In the present application, the expression “time-averaged irradiance” refers to the irradiance averaged over time. For example, the “time-averaged irradiance” at a target position (i.e., point) refers to the irradiance averaged overtime at the target position. As another example, the “time-averaged irradiance” over a target region refers to the “time-averaged irradiance” at the representative irradiated positions on the target region. Specifically, a “time-averaged irradiance” of 30-60 mW/cm² over a target region means that the time-averaged irradiance at each representative irradiated position on the target region, such as but not limited to the position corresponding to the center of the irradiation panel, fluctuates within the range of 30-60 mW/cm².

The expression “spatiotemporally averaged irradiance” over a target region is intended to mean a parameter obtained by performing an averaging operation over the surface (over the surface area) of the target region based on the time-averaged irradiances (at the representative irradiated positions on the target region), that is, the irradiance obtained by performing averaging operations over both the surface (spatio-) and time (temporally).

As long as the near-infrared light irradiation delivered to the subject's head satisfies the coordinated irradiation condition of the irradiated surface area ratio and the delivered radiant power level, near-infrared light with sufficient radiant power can be delivered to a sufficient region of the brain tissue within a unit time. For example, but not by way of limitation, this works for the black thick hair wearing the light guide comb as described in the applicant's patent application No. PCT/CN2021/126700, or for the light thick hair not wearing the light guide comb, or for black sparse hair no matter wearing the light guide comb or not. For the locally focused stimulation protocol, the irradiated surface area ratio may be as low as 30%, while for the multi-region balanced stimulation protocol, the irradiated surface area ratio may be as high as 65% or even more. For both locally focused stimulation protocol and multi-region balanced stimulation protocol, the irradiated surface area ratio within this range, in coordination with the matched delivered radiant power level, can achieve the “modulation” and “activation” of a sufficient proportion of cell populations. After a sufficient proportion of cell populations are “modulated” and “activated”, such cell populations not only exhibit their own change responses that inhibit AD, but also propagate and diffuse such change responses to other cell populations along the spatial trajectory of AD progression, thereby achieving a comprehensive AD inhibition effect throughout the entire brain. In this way, it can not only significantly reduce Aβ plaques aggregated in the neocortex, significantly reduce abnormal aggregation of tau protein in the neocortex, hippocampus, and even the limbic cortex, but also inhibit and eliminate AD-specific pathological lesions of cell populations throughout the entire brain, thereby effectively inhibiting the progression of the disease course of AD.

In some embodiments, adaptive coordinated irradiation conditions may be provided for the locally focused stimulation protocol and the multi-region balanced stimulation protocol.

Specifically, the irradiated surface area ratio of the locally focused stimulation protocol is smaller than that of the multi-region balanced stimulation protocol, and the matched delivered radiant power level of the locally focused stimulation protocol is also higher than that of the multi-region balanced stimulation protocol. For example, as the locally focused stimulation protocol, in case that the irradiated surface area ratio is from 30% to 40%, the spatiotemporally averaged irradiance over the irradiated surface of the subject's head is required to be 117 mW/cm² or more; and in case that the irradiated surface area ratio is between 40% and 65%, the spatiotemporally averaged irradiance over the irradiated surface of the subject's head is 110 mW/cm² or more. In this way, it is possible to break through the local limitation of cell populations, sufficiently “modulate” and “activate” the cell populations, and propagate and diffuse the change responses that inhibit AD to other cell populations in a broader region along the spatial trajectory of AD progression.

The multi-region balanced stimulation protocol requires an average coordinated energy delivery of 2750 W·% to 14100 W·% to the subject's head, wherein the average coordinated energy delivery is a product of the percentage value of the irradiated surface area ratio and the average total power incident upon (i.e., delivered to) the head, so the unit is W·%. For example, if the irradiated surface area ratio is 65%, its percentage value is 65, and the average coordinated energy delivery is the average total delivered radiant power multiplied by 65 W·%. As the affected cell populations are distributed more widely, for example, in multiple brain regions, multiple brain functional networks, etc., by means of receiving the above average coordinated energy delivery, the cell populations can be appropriately “modulated” and “activated”, and the change responses for inhibiting AD can be propagated and diffused to other cell populations in a wide range along the spatial trajectory of AD progression.

Compared with the prior art, the beneficial effects of the embodiments of the present application are as follows. The phototherapy apparatus can adopt various structures, and is not limited to whether the near-infrared irradiation units are ordinary LEDs or low-energy laser diodes. It ensures that the irradiation of near-infrared light delivered to the subject's head satisfies the coordinated irradiation condition of the irradiated surface area ratio and the delivered radiant power level, that is, a sufficient composite energy delivery of time-averaged radiant power and irradiated surface area ratio is administered to the subject's head, thereby embedding the population response characteristics of cell sub-populations, the action mechanism of cerebral functional networks, and the whole-brain progression characteristics of AD therein. By means of sufficient composite energy delivery of near-infrared light to the subject's head, no matter the locally focused stimulation protocol or the multi-region balanced stimulation protocol is adopted, it can achieve the “modulation” and “activation” of a sufficient proportion of cell populations. After a sufficient proportion of cell populations are “modulated” and “activated”, such cell populations not only exhibit their own change responses that inhibit AD, but also propagate and diffuse such change responses to other cell populations along the spatial trajectory of AD progression, thereby achieving a comprehensive AD inhibition effect throughout the entire brain. In this way, the present application can not only significantly reduce Aβ plaques aggregated in the neocortex, significantly reduce abnormal aggregation of tau protein in the neocortex, hippocampus, and even the limbic cortex, but also inhibit and eliminate AD-specific pathological lesions of cell populations throughout the entire brain, thereby effectively inhibiting the progression of the disease course of AD. The inhibitory effect of the phototherapy apparatus of the present application on the AD progression has also been validated in clinical trials. Not only did the subjects exhibit a significant improvement in cognitive level during the period of phototherapy administration, but also a series of biochemical reactions caused by light irradiation continued to trigger the inhibitory effect during the continuous period after the cessation of phototherapy. This not only maintains the inhibitory effect on AD to a certain extent, but also continuously promotes the inhibitory effect on AD, with the cognitive level still being maintained or improved, without deterioration and regression (as will be described in detail below).

Both the foregoing general description and the following detailed description are exemplary and illustrative only and are not intended to limit the present invention.

The summary of various implementations or examples of the technology described in the present invention is not a comprehensive disclosure of the full scope or all features of the disclosed technology.

### BRIEF DESCRIPTION OF THE DRAWINGS

In figures that are not necessarily drawn to scale, the same reference numerals may describe similar components in different figures. The same reference signs with suffixes or different suffixes may denote different examples of similar components. The figures generally show various embodiments by way of example rather than limitation, and are used together with the description and the claims to describe the embodiments of the invention. As proper, the same reference sign may be used throughout the drawings to denote the same part. Such embodiments are illustrative, and are not intended to be exhaustive or exclusive embodiments of the present device or method.

FIG. 1(a) shows a schematic diagram of a phototherapy apparatus according to a first embodiment of the present application;

FIG. 1(b) shows a structural schematic diagram of a head-worn device of the phototherapy apparatus according to the first embodiment of the present application;

FIG. 2(a) shows a schematic diagram of a phototherapy apparatus according to a second embodiment of the present application;

FIG. 2(b) shows a schematic diagram of a phototherapy apparatus according to a third embodiment of the present application;

FIG. 3(a) to FIG. 3(f) show schematic diagrams of a reference head phantom of a population of treatment subjects according to an embodiment of the present application;

FIG. 4(a) shows a front view of a reference head phantom as an example of a subject's head according to a fourth embodiment of the present application, on which the electrode positions of the international 10-10 standard system, the total boundary line of the reference calvarial region, and the boundary lines among the anterosuperior cranial region, the left lateral cranial region and the right lateral cranial region are shown;

FIG. 4(b) shows a left side view of the reference head phantom as an example of a subject's head according to the fourth embodiment of the present application, on which the electrode positions of the international 10-10 standard system, the total boundary line of the reference calvarial region, and the boundary lines among the anterosuperior cranial region, the left lateral cranial region, the cranial vertex region and the posterior cranial region are shown;

FIG. 4(c) shows a right side view of the reference head phantom as an example of a subject's head according to the fourth embodiment of the present application, on which the electrode positions of the international 10-10 standard system, the total boundary line of the reference calvarial region, and the boundary lines among the anterosuperior cranial region, the right lateral cranial region, the cranial vertex region and the posterior cranial region are shown;

FIG. 4(d) shows a top view of the reference head phantom as an example of a subject's head according to the fourth embodiment of the present application, on which the electrode positions of the international 10-10 standard system, the total boundary line of the reference calvarial region, and the boundary lines among the anterosuperior cranial region, the cranial vertex region, the left lateral cranial region, the right lateral cranial region and the posterior cranial region are shown;

FIG. 4(e) shows a rear view of the reference head phantom as an example of a subject's head according to the fourth embodiment of the present application, on which the electrode positions of the international 10-10 standard system, the total boundary line of the reference calvarial region, and the boundary lines among the cranial vertex region, the left lateral cranial region, the right lateral cranial region and the posterior cranial region are shown;

FIG. 5(a) shows a schematic diagram of the anterosuperior cranial region of a reference head phantom as an example of a subject's head according to a fifth embodiment of the present application;

FIG. 5(b) shows a schematic diagram of the anterosuperior cranial region of a reference head phantom as an example of a subject's head according to a sixth embodiment of the present application;

FIG. 5(c) shows a schematic diagram of the anterosuperior cranial region of a reference head phantom as an example of a subject's head according to a seventh embodiment of the present application;

FIG. 6(a) shows an exemplary diagram of an irradiated region according to an eighth embodiment of the present application;

FIG. 6(b) shows an exemplary diagram of an irradiated region according to a ninth embodiment of the present application;

FIG. 6(c) shows an exemplary diagram of an irradiated region according to a tenth embodiment of the present application;

FIG. 6(d) shows an exemplary diagram of an irradiated region according to an eleventh embodiment of the present application;

FIG. 7 shows a bottom view of a headgear of a phototherapy apparatus according to a twelfth embodiment of the present application;

FIG. 8 shows a structural schematic diagram of an arrangement of irradiation panels according to a thirteenth embodiment of the present invention;

FIG. 9 shows a structural schematic diagram of an arrangement of irradiation panel frame according to a fourteenth embodiment of the present invention;

FIG. 10 shows a schematic flowchart of a clinical trial on an AD patient using the phototherapy apparatus according to the embodiment of the present application;

FIG. 11(a) shows a trend graph of ADAS-Cog scale scores of the sham control group before near-infrared phototherapy, during near-infrared phototherapy, and after cessation of near-infrared phototherapy;

FIG. 11(b) shows a trend graph of ADAS-Cog scale scores of the intervention group before near-infrared phototherapy, during near-infrared phototherapy, and after cessation of near-infrared phototherapy;

FIG. 12(a) shows a trend graph of MMSE scale scores of the sham control group before near-infrared phototherapy, during near-infrared phototherapy, and after cessation of near-infrared phototherapy;

FIG. 12(b) shows a trend graph of MMSE scale scores of the intervention group before near-infrared phototherapy, during near-infrared phototherapy, and after cessation of near-infrared phototherapy.

### DETAILED DESCRIPTION

In order to make the objects, technical solutions and advantages of the embodiments of the present invention clearer, the technical solutions of the embodiments of the present invention will be clearly and completely described below with reference to the accompanying drawings of the embodiments of the present invention. Obviously, the described embodiments are part of the embodiments of the present invention, rather than all of the embodiments. Based on the described embodiments of the present invention, all other embodiments obtained by a person of ordinary skill in the art without creative labor shall fall within the protection scope of the present invention.

Unless otherwise defined, technical or scientific terms used in the present invention shall have the ordinary meaning as understood by a person of ordinary skill in the art to which the present invention belongs. The terms “first”, “second” and the like used in the present invention do not denote any order, quantity or importance, but are only used to distinguish different components. The terms “comprise” or “include” and the like mean that the element or item preceding such a term covers the element or item listed after the word and its equivalents, without excluding other elements or items. The terms “connect” or “couple” and the like are not limited to physical or mechanical connections, but may include electrical connections, whether direct or indirect. Words such as “lower”, “upper”, “left”, and “right” are only used to indicate relative positional relationships, and when the absolute position of the described object changes, the relative positional relationship may also change accordingly.

To keep the following description of the embodiments of the present invention clear and concise, the present invention omits detailed descriptions of known functions and known components.

The present application aims to provide a phototherapy apparatus for treating Alzheimer's disease and associated conditions. In the present application, the expression “for treating Alzheimer's disease and associated conditions” is intended to mean alleviating, inhibiting, terminating, or even reversing the progression of the disease course of Alzheimer's disease and its associated conditions. The so-called associated conditions of Alzheimer's disease are intended to mean that the subject has not yet exhibited obvious clinical symptoms of AD, but has certain pathological or physiological phenomena associated with AD that have a certain probability of developing into AD. The so-called disease course of Alzheimer's disease and its associated conditions is intended to include the process in which the subject has already exhibited clinical symptoms of AD and the subsequent development process, as well as the process in which the subject has not yet exhibited obvious clinical symptoms of AD, but has some pathological or physiological phenomena associated with AD that have a certain probability of developing into AD. That is to say, the expression “for treating Alzheimer's disease and associated conditions” used in the present application includes the treatment of Alzheimer's disease at various stages of its disease course (from MCI to severe dementia), and also includes the treatment of other conditions associated with Alzheimer's disease. Specifically, according to the diagnostic criteria for AD in 2018 by the US National Institute on Aging and Alzheimer's Association (NIA-AA), based on the examination results of β-amyloid (Aβ) and tau in the brain or cerebrospinal fluid of the population, as well as cranial MRI and FDG-PET, biomarkers may be classified into 4 categories and cognitive function may be further divided into 6 levels. Level 1 is manifested as normal objective cognitive neuropsychological tests, no cognitive complaints, no neurobehavioral symptoms, no report of cognitive decline or neurobehavioral symptoms from informants, and no follow-up test evidence of cognitive decline. Level 2 includes subjective cognitive decline (SCD), objective subtle cognitive decline (Obj-SCD), and neurobehavioral symptoms. Level 1 and Level 2 are collectively referred to as the preclinical stage. Level 3 is abnormal or impaired performance on objective cognitive tests that does not amount to dementia, namely MCI. Levels 4-6 are mild, moderate and severe dementia, respectively. For each of these 6 levels, if there is a positive test result of biomarkers, it may belong to the disease course of Alzheimer's disease as defined in the present application. Furthermore, for subjects carrying genes associated with the risk of AD, such as APOEε4, ABCA7, CLU, CR1, PICALM, PLD3 and TREM2 or the like, but whose current test result is negative AB, even if the cognitive function is at Level 1, the process of performing medical intervention on them to reduce the risk of suffering from AD or to slow down the process of developing into AD may also be regarded as “for treating associated conditions of Alzheimer's disease” in the present application.

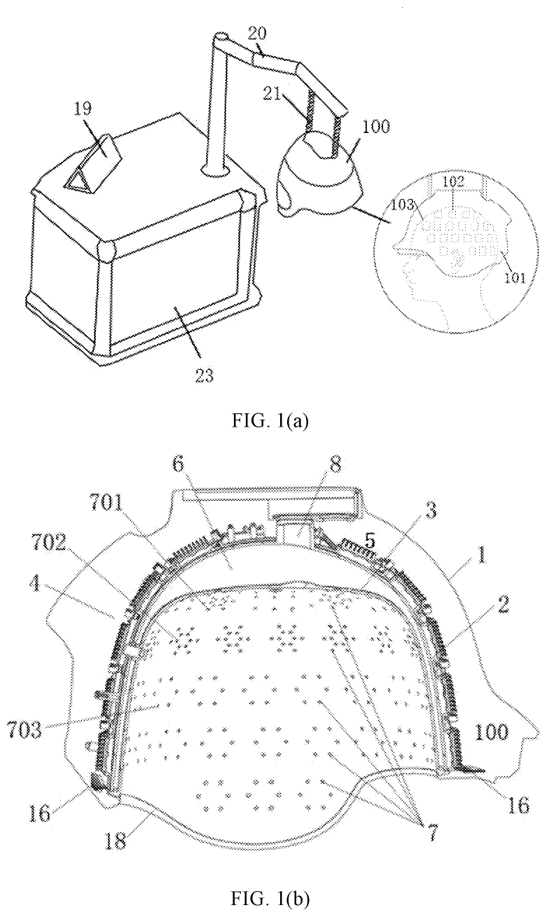

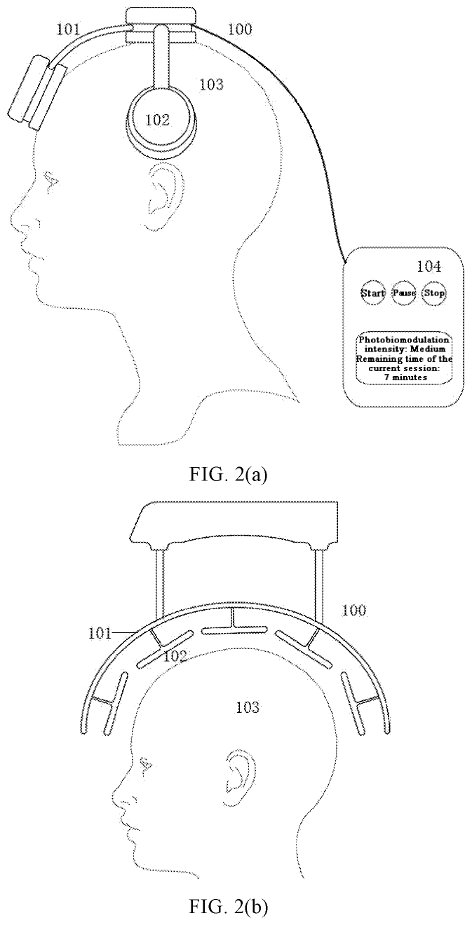

The phototherapy apparatus includes a support mechanism 101 and an array of near-infrared irradiation units 102. The support mechanism 101 is configured to form an accommodation space for a subject's head 103 and to support the array of near-infrared irradiation units 102. The array of near-infrared irradiation units 102 is configured to emit near-infrared light into the accommodation space. The support mechanism 101 and the array of near-infrared irradiation units 102 may adopt various structures as needed, for example, see FIG. 1(a), FIG. 2(a) and FIG. 2(b), which will be described in detail below, but the structure of the phototherapy apparatus is not limited thereto.

When the subject's head 103 is positioned in place within the accommodation space, the near-infrared light irradiation delivered to the subject's head satisfies a coordinated irradiation condition of an irradiated surface area ratio and a delivered radiant power level. The irradiated surface area ratio is a ratio of an irradiated surface area to a surface area of a reference calvarial region 401. As used herein, the so-called “in place” as used in the present application is intended to mean that the subject's head 103 is set up at a desired treatment position in the phototherapy apparatus, that is, has a desired spatial position and spatial orientation. Usually, the phototherapy apparatus can be activated when the subject's head 103 is positioned “in place”. For example, when positioned in place, the center of gravity of the subject's head 103 can be aligned with the center of the accommodation space, and the central axes thereof in the front-rear direction are aligned with each other. For another example, when positioned in place, the subject's head 103 can be centered in the accommodation space, with the front-rear distances from the front and rear walls of the accommodation space being substantially uniform, and the left-right distances from the left and right walls of the accommodation space also being substantially uniform. For another example, taking the phototherapy headgear shown in FIG. 1(a) as an example, when positioned in place, the supraorbital ridges of the subject's head 103 may be flush with the front edge of the headgear, and the subject's head 103 is located in a centered position within the headgear, so that the front and rear distances from the irradiation surface of the inner housing are substantially uniform, and the left and right distances from irradiation surface of the inner housing are also substantially uniform.

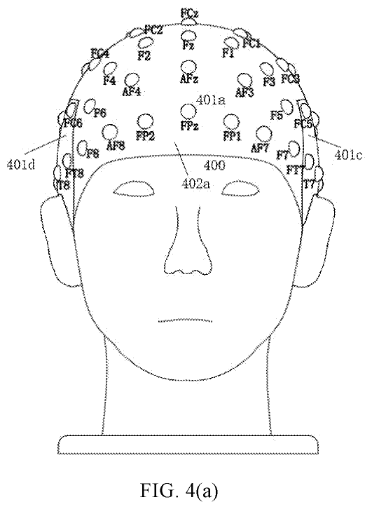

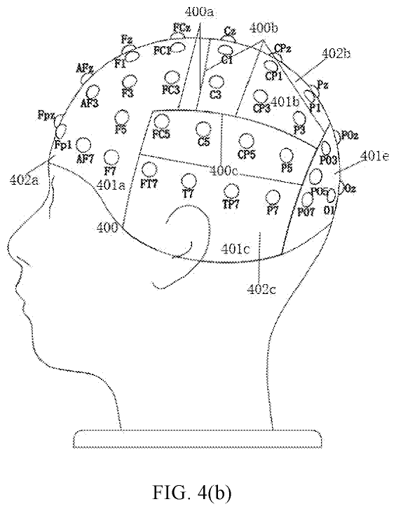

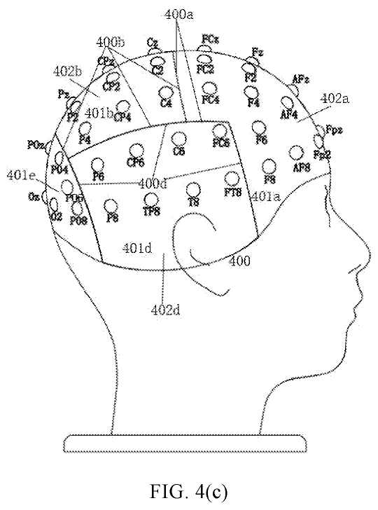

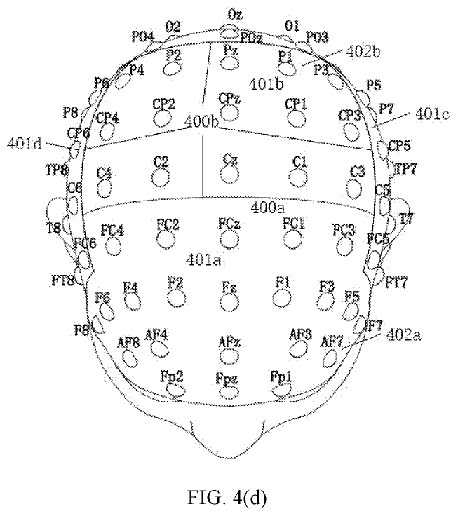

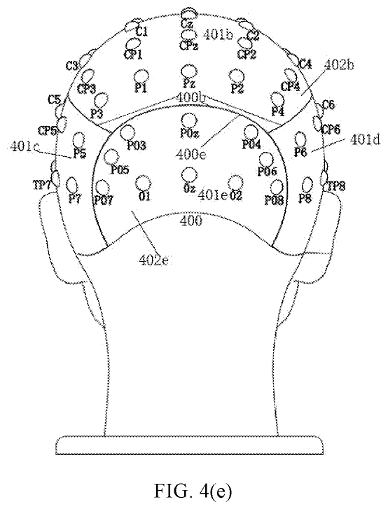

The surface area of the reference calvarial region is the external surface area of a surface of the subject's head within a total boundary line 400, the total boundary line 400 extends from a Glabella point of the subject's head along supraorbital ridges, passes through preauricular points on both sides, curves posteriorly around the head to pass between the inion and the electrode positions O1, OZ, and O2 of the international 10-10 standard system (i.e., the international 10-10 standard EEG placement system), and then converges, as shown in FIG. 4(a), FIG. 4(b), FIG. 4(c), and FIG. 4(e). The irradiated surface area ratio may be in the range of 30% (i.e., may be as low as 30%, which may be referred to as a locally focused stimulation protocol) to 65% or more (i.e., may be up to 65% or even more, which may be referred to as a multi-region balanced stimulation protocol).

The inventors have creatively found through clinical trials, including case studies and experiments on a target population within a certain range, that the effect of treating Alzheimer's disease and its associated conditions by irradiating near-infrared light of the same wavelength cannot be determined solely by the irradiance. Both the delivered radiant power level that can be delivered to the brain tissues and the irradiated surface area ratio are important factors, and they determine the effect in a coordinated manner. Furthermore, the energy attenuation of near-infrared light passing through the skull can be measured. For example, see Jagdeo J R et al., Transcranial red and near infrared light transmission in a cadaveric model. PLoS One 2012; 7:e47460, at a depth of 10 mm (an approximate thickness of cadaver skull and its intact soft tissue), the transmission percentage of LED light with 830 nm wavelength is 0.9% for the temporal lobe, 2.1% for the frontal lobe, and 11.7% for the occipital lobe. In addition, the light absorption and attenuation caused by hair of various thicknesses, amounts and colors can also be measured. That is to say, considering the attenuation on the transmission path from the irradiation emergent surface to the target brain tissue, by making the near-infrared light irradiation delivered to the subject's head satisfy the coordinated irradiation condition of the irradiated surface area ratio and the delivered radiant power level, it is possible to deliver near-infrared light with sufficient radiant power per unit time to a sufficient region of the subject's head, and thereby deliver near-infrared light with still sufficient radiant power per unit time after attenuation on the transmission path to a sufficient region of the brain tissue, so as to achieve an optimized therapeutic effect for Alzheimer's disease and its associated conditions. The optimization of the therapeutic effect has also been validated by clinical trials (as will be described in detail below), and the phototherapy apparatus of the present application indeed exhibits an excellent “runtime per charge” (i.e., sustaining) capability in inhibiting the progression of disease courses of AD. Specifically, not only the subjects exhibit a significant improvement in cognitive level during the period of phototherapy administration, but also their cognitive level is maintained or improved during the continuous period after the cessation of phototherapy, and the subjects do not experience any adverse reactions. The main action mechanism for this finding is estimated as follows, but the exact action mechanism needs to be confirmed and verified through more experiments.

AD is a whole-brain disease, and its pathological changes are not limited to a specific region of the brain, but gradually spread from some regions to the entire brain, affecting multiple brain regions and neural networks. For example, the accumulation of Aβ in the brain follows a specific spatial trajectory, starting from the default mode network (DMN) regions and gradually spreading to other low-order sensorimotor regions.

In addition, single-cell transcriptomics studies have revealed cell type-specific changes in AD, that is, different cell populations have different population responses associated with AD, and the cell populations include but not limited to astrocytes, microglia, oligodendrocytes, neurons, vascular cells, peripheral glial cells, extracellular matrix, etc. Specifically, subpopulations of astrocytes have been found to be associated with cognitive decline, and they play a role in regulating the effect of tau protein on cognitive function. Different subpopulations of microglia are associated with the pathogenesis of AD, wherein some subpopulations promote Aβ protein lesions, while others regulate the effect of Aβ protein on tau protein lesions. The specific responses of oligodendrocytes in AD are associated with disease progression, such as subpopulation-specific transcriptional changes. AD affects specific neuronal subpopulations, such as those in the hippocampus and cerebral cortex regions, which are closely associated with AD. Vascular cells include vascular endothelial cells and perivascular cells, which play a role in vasculopathy in AD. Peripheral glial cells are associated with APOE expression and play a role in neurodegeneration in AD. Changes in the extracellular matrix are also associated with the progression of AD, affecting intercellular interactions and signal transmission.

As long as the near-infrared light irradiation delivered to the subject's head satisfies the coordinated irradiation condition of the irradiated surface area ratio and the delivered radiant power level, near-infrared light with sufficient radiant power can be delivered to a sufficient region of the brain tissue within a unit time. For the locally focused stimulation protocol, the irradiated surface area ratio may be as low as 30%, while for the multi-region balanced stimulation protocol, the irradiated surface area ratio may be up to 65% or more. The irradiated surface area ratio within this range, in coordination with the matched delivered radiant power level, can achieve the “modulation” and “activation” of a sufficient proportion of cell populations. After a sufficient proportion of cell populations are “modulated” and “activated”, they not only exhibit change responses that inhibit AD, but also propagate and diffuse this change responses to other cell populations along the spatial trajectory of AD progression, thereby achieving a comprehensive AD inhibitory effect throughout the entire brain. In this way, it can not only significantly reduce Aβ plaques aggregated in the neocortex, significantly reduce abnormal aggregation of tau protein in the neocortex, hippocampus, and even the limbic cortex, but also inhibit and eliminate AD-specific pathological lesions of cell populations throughout the entire brain, thereby effectively inhibiting the progression of the disease course of AD and associated conditions. Furthermore, during the continuous period after the cessation of phototherapy, a series of biochemical reactions caused by near-infrared light irradiation continue to trigger the inhibitory effect, which not only maintains the inhibitory effect on AD to a certain extent, but also continuously promotes the inhibitory effect on AD without deterioration and regression.

In some embodiments, adaptive coordinated irradiation conditions may be provided for the locally focused stimulation protocol and the multi-region balanced stimulation protocol.

Specifically, the irradiated surface area ratio of the locally focused stimulation protocol is smaller than that of the multi-region balanced stimulation protocol, and the matched delivered radiant power level of the locally focused stimulation protocol is also higher than that of the multi-region balanced stimulation protocol. For example, as the locally focused stimulation protocol, in case that the irradiated surface area ratio is from 30% to 40%, the spatiotemporally averaged irradiance over the irradiated surface of the subject's head is required to be 117 mW/cm² or more; and in case that the irradiated surface area ratio is between 40% and 65%, the spatiotemporally averaged irradiance over the irradiated surface of the subject's head is 110 mW/cm² or more. In this way, it is possible to break through the local limitation of cell populations, sufficiently “modulate” and “activate” the cell populations, and propagate and diffuse the change responses that inhibit AD to other cell populations in a broader region along the spatial trajectory of AD progression. Note that in the present application, the description of percentage ranges is defined as follows. The expression “between A % and B %” is intended to include the percentages between A % and B %, excluding both endpoints A % and B %. The expression “from A % to B %” is intended to include the percentages between A % and B %, including both endpoints A % and B %.

The multi-region balanced stimulation protocol requires an average coordinated energy delivery of 2750 W·% to 14100 W·%, where the average coordinated energy delivery is the product of the irradiated surface area ratio and the average total power incident upon (i.e., delivered to) the subject's head, so the unit is W·%. As the affected cell populations are distributed more widely, for example, in multiple brain regions, multiple brain functional networks, etc., by administering the above average coordinated energy delivery, the cell populations can be appropriately “modulated” and “activated”, and the change responses for inhibiting AD can be propagated and diffused to other cell populations in a wide region along the spatial trajectory of AD progression. Note that for various coordinated irradiation conditions of the irradiated surface area ratio and the delivered radiant power level, the spatiotemporally averaged irradiance over the irradiated surface of the subject's head is 230 mW/cm² or less to avoid thermal damage to tissue cells.

The phototherapy apparatus and head-worn device 100 thereof according to the first embodiment of the present application are shown in FIG. 1(a). The support mechanism 101 is configured as a headgear, which maintains an appropriate gap from the subject's head 103 when the head is accommodated therein, allowing the subject's head 103 to move. This loose and open design of the headgear has no sense of restraint on the patient's head, and is particularly friendly for the elderly who are emotionally agitated, anxious, resistant, or even afraid of confined or crowded spaces, thereby significantly improving the treatment compliance of patients with AD. The array of near-infrared irradiation units 102 can be formed as irradiation panels and assembled and fixed inside the headgear.

The phototherapy apparatus may further include a user terminal 19 configured for interactive operation by a user. The user terminal 19 may be configured with a computer storage medium having executable instructions stored thereon. When the computer-executable instructions are executed by a processor, various interactive steps with the user can be implemented. The storage medium may include read-only memory (ROM), flash memory, random access memory (RAM), dynamic random access memory (DRAM) such as synchronous DRAM (SDRAM) or Rambus DRAM, static memory (e.g., flash memory), static random access memory, etc., on which computer-executable instructions may be stored in any format. In some embodiments, the user terminal 19 is further configured to receive a user's confirmation operation for a proposed infrared light therapeutic protocol; after receiving the confirmation operation, irradiation is performed according to the confirmed infrared light therapeutic protocol.

Specifically, a controller (not shown) for controlling irradiation may be located on the user terminal 19, or on the head-worn device 100, or on a host device different from the user terminal 19 and the head-worn device 100. The controller can be implemented by various processors, and may be a processing device including one or more general-purpose processing devices, such as a microprocessor, a central processing unit (CPU), a graphics processing unit (GPU), etc., or one or more special-purpose processing devices, such as an application-specific integrated circuit (ASIC), a field-programmable gate array (FPGA), a digital signal processor (DSP), a system on chip (SoC), etc. Preferably, most of the computation and processing is concentrated on the user terminal 19 to reduce the computing load and hardware and software costs of the head-worn device. The head-worn device 100 is suspended to a bracket 20 via an elastic member 21, and the bracket 20 adopts a three-section freely pivoting structure to facilitate flexible adjustment of the position of the head-worn device 100. In some embodiments, the phototherapy apparatus is further equipped with a refrigerator (e.g., compressor) 23 to introduce cooled gas into the phototherapy apparatus to achieve sufficient and comfortable cooling around the subject's head 103, for example, to stabilize the temperature at about 43° C., or even about 41° C.

FIG. 1(b) shows an exemplary configuration of this loose head-worn device 100. As shown in FIG. 1(b), the head-worn device 100 is implemented as a headgear, which includes an outer housing 1, a middle housing 2 and an inner housing 3 arranged in sequence from outside to inside. An irradiation panel accommodating cavity 4 is formed between the outer housing 1 and the middle housing 2, in which a plurality of irradiation panels 5 (corresponding to the array of near-infrared irradiation units 102 in FIG. 1(a)) are disposed. A cooled gas cavity 6 is formed between the middle housing 2 and the inner housing 3, and the inner housing 3 encloses inwardly to form an accommodation space. Wherein, a plurality of ventilation holes 7 are opened in the inner housing 3, so that the cooled gas passing through the cooled gas cavity 6 can enter the accommodation space through the ventilation holes 7. The ventilation holes 7 may be opened in a first region of the inner housing 3 close to the top of the head and a second region below the first region. The second region is provided with a plurality of ventilation hole units arranged in layers from top to bottom, wherein the arrangement of the upper-layer ventilation hole unit is different from that of the lower-layer ventilation hole unit, so as to achieve cooling effect with respect to the individual regions of the patient's head and improve the synchronicity and uniformity of cooling. During the phototherapy process, the patient's entire head experiences a more uniform temperature distribution and higher comfort.

It can be seen that the inner housing 3 is the housing layer closest to the patient's head when worn, the cooled gas cavity 6 is formed between the middle housing 2 and the inner housing 3, and both the inner housing 3 and the middle housing 2 are configured to be light transmissive. In this way, the near-infrared light (for example, with a wavelength of 800-850 nm) emitted from the irradiation panel 5 can sequentially pass through the light transmissive middle housing 2 and inner housing 3 into the accommodation space, and thus sufficient irradiation is delivered to the patient's head.

In some embodiments, the inner housing 3 includes two parts: a first region close to the top of the head and a second region located below the first region. The first region and the second region may be provided with ventilation holes 7. As shown in FIG. 1(b), the middle housing 2 is provided with a cooled gas inlet 8 interconnected to the cooled gas cavity 6, a ring of ventilation holes may be provided at least on the outer edge of the first region, and a cavity may be reserved for the top of the head for placing a protective pad therein. After the cooled gas enters the cooled gas cavity 6 through the cooled gas inlet 8, it passes through the ventilation holes 7 and then blows towards the patient's head, to ensure that the patient's pericephalic and vertex regions can experience a good cooling effect. In addition, the above structure enables the cooled gas to flow from the upper part of the patient's head downward, facilitate to improve the heat exchange efficiency and temperature uniformity in the accommodation space.

In this embodiment, the cooled gas inlet 8 of the cooled gas cavity 6 is provided in the first region, and is closer to the rear of the inner housing 3 than to the front of the inner housing 3, wherein the front of the inner housing 3 is the direction of the headgear corresponding to the forehead, and the rear of the inner housing 3 is the direction of the headgear corresponding to the afterbrain. In this way, the forehead region will not be excessively cooled, while the afterbrain, which absorbs more irradiation energy due to a larger amount of hair and thus generates more heat, can also experience an enhanced cooling effect, thereby improving the patient's comfort during phototherapy. This is only an example, in some embodiments, the cooled gas inlet 8 is kept at a substantially the same distance from the front end of the inner housing 3 (i.e., the edge position at the front of the inner housing 3) and the rear end of the inner housing 3 (i.e., the edge position at the rear of the inner housing 3), so as to avoid untimely and non-uniform cooling in the regions caused by a relatively large difference in the two distances.

In some embodiments, in order to deliver cooled gas more uniformly into the accommodation space, the headgear may include a plurality of cooled gas inlets 8, which are distributed at different positions of the inner housing. The position setting of the cooled gas inlets 8 may be correspondingly designed and adjusted according to the specific structure of the headgear and the layout of the phototherapy irradiation panels.

In some embodiments, the second region is provided with a plurality of ventilation hole units in layers from top to bottom, the arrangement of the upper-layer ventilation hole units 701 is different from that of the lower-layer ventilation hole units 703, and the arrangement of the upper-layer ventilation hole units 701 is the same as that of the middle-layer ventilation hole units 702. For example, the number of ventilation holes on a single lower-layer ventilation hole unit 703 is less than that on a single upper-layer ventilation hole unit 701 or middle-layer ventilation hole unit 702. For another example, ventilation holes 7 with a relatively lower arrangement density than that of ventilation holes 7 in each upper-layer ventilation hole unit 701 are provided between adjacent upper-layer ventilation hole units 701. Such configuration enables the upper-layer ventilation hole units 701 and the middle-layer ventilation hole units 702 to release more cooled gas compared to the lower-layer ventilation hole units 703.

In some embodiments, the upper-layer ventilation hole units 701 and the middle-layer ventilation hole units 702 may each be composed of ventilation holes 7 uniformly distributed on an inner ring and ventilation holes 7 uniformly distributed on an outer ring. For example, 6 ventilation holes 7 are uniformly distributed on the inner ring and 6 ventilation holes 7 are uniformly distributed on the outer ring, while the lower-layer ventilation hole unit 703 may be composed of only a single ring of uniformly distributed ventilation holes 7, for example, only 6 ventilation holes 7 are uniformly distributed.

The refrigerator 23 shown in FIG. 1(a), the cooled gas inlet 8, the cooled gas cavity 6, the ventilation holes 7, and the interconnection between the accommodation space and the external environment shown in FIG. 1(b) cooperatively constitute a cooling mechanism. With this cooling mechanism, when the time-averaged total irradiation power incident upon the subject's head 103 reaches 27.5-120 W and a single continuous irradiation duration (session duration) reaches 30 minutes, the temperature of air in a space adjacent to, but not in contact with, the subject's head does not exceed 41° C., that is, the temperature of the ambient air in which the subject's head 103 is immersed does not exceed 41° C.

In some embodiments, the ventilation holes 7 are at least provided at positions corresponding to the irradiation panels 5. During the phototherapy of the patient, at the positions where the irradiation panels 5 irradiate the patient's skin, heat is more likely to accumulation due to the high intensity of light irradiation. By means of providing the ventilation holes 7 at least at the positions on the inner housing 3 corresponding to the irradiation panels 5, the irradiation of the irradiation panels 5 on the patient's skin can be prevented from causing excessive heat accumulation.

In some embodiments, the density of the ventilation holes 7 at the positions corresponding to the irradiation panels 5 is greater than the density of the ventilation holes 7 at the positions not corresponding to the irradiation panels 5, so as to further balance the temperature at the positions corresponding to the irradiation panels 5 and other positions, enhance the air permeability of the accommodation space, and improve the air heat exchange rate in the accommodation space.

As an example, the loose head-worn device 100 shown in FIG. 1(b) is particularly suitable for implementing the multi-region balanced stimulation protocol. Of course, through flexible control of the irradiation panels 5, the loose head-worn device 100 can also implement the locally focused stimulation protocol as needed.

FIG. 2(a) shows a schematic diagram of a phototherapy apparatus according to a second embodiment of the present application. As shown in FIG. 2(a), the phototherapy apparatus includes a head-worn device 100 and a portable control terminal 104 which are communicatively connected.

The support mechanism 101 adopts a hollow frame pressed against the subject's head 103, and discrete irradiation modules formed of the array of near-infrared irradiation units 102 are fixedly mounted on the hollow frame. Under the action of the hollow frame, the irradiation modules are brought into close abutment against the forehead part, cranial vertex part, supra-auricular part and the like of the subject's head, so as to emit near-infrared light to these parts.

As an example, the control terminal 104 may be provided with operation buttons, such as but not limited to a start button, a pause button, and a stop button, so that an operator can control the phototherapy process via the buttons. The control terminal 104 may also be provided with a display to present operation information of the phototherapy process to the operator, such as but not limited to photobiomodulation intensity and remaining time of the current session.

Specifically, for example, the phototherapy apparatus with the structure shown in FIG. 2(a) is particularly suitable for implementing the locally focused stimulation protocol, and/or for the therapy of earlier stages of AD course (such as during MCI and mild dementia) and for preventive medical intervention for AD. Furthermore, the phototherapy apparatus with the structure shown in FIG. 2(a) has good portability, allowing the subject to use it conveniently while at home or traveling, thereby ensuring the frequency and continuity of phototherapy.

FIG. 2(b) shows a schematic diagram of a phototherapy apparatus according to a third embodiment of the present application, and its control and processing terminal may refer to those in the previous other embodiments, and is not shown here to simplify the description. Different from the head-worn devices 100 shown in FIG. 1(b) and FIG. 2(a), the support mechanism 101 forms a significantly wider, arch-shaped or umbrella-shaped accommodation space, and is located farther from the subject's head 103. Moreover, for example, the support mechanism 101 is rigidly mounted to a bracket or a wall, and discrete irradiation modules formed of the array of near-infrared irradiation units 102 are mounted on the inner wall of the support mechanism 101.

In some embodiments, the coordinated irradiation condition of the irradiated surface area ratio and the delivered radiant power level may characterize a correlation between the irradiated surface area ratio and the spatiotemporally averaged irradiance over the irradiated surface of the subject's head, such as the coordinated irradiation condition adopted in the locally focused stimulation protocol in case that the irradiated surface area ratio is from 30% to 40% or between 40% and 65% as described above; or a product of the average total radiant power incident upon the subject's head and the total irradiated surface area ratio, such as the coordinated irradiation condition adopted in the multi-region balanced stimulation protocol in case that the irradiated surface area ratio is 65% or more as described above. In other embodiments, the coordinated irradiation condition of the irradiated surface area ratio and the delivered radiant power level may be defined differently as needed, for example, an integral of the radiant power incident upon (i.e., irradiated onto and delivered to) each partition of the subject's head with respect to the irradiated surface area ratio of each partition, or a distribution of the product of the radiant power incident upon (i.e., irradiated onto) each partition of the subject's head and the irradiated surface area ratio of each partition, etc. Refining the coordinated irradiation condition based on the partition distribution can more effectively identify situations in which the corresponding radiant power incident upon a partition or irradiance (i.e., radiant power density) over the partition is too low when the deviation in irradiation power among various brain regions is relatively large, ensuring that there is no dead zone in the “modulation” and “activation” of cell populations.

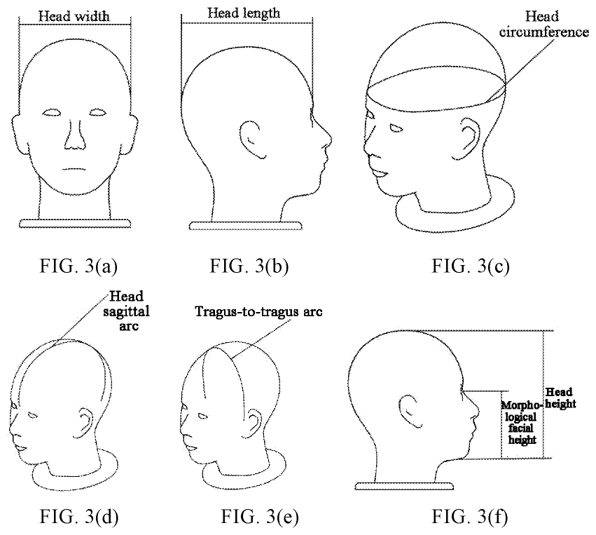

In some embodiments, the subject's head may comprise either the head of a patient or a reference head phantom of a population of treatment subjects. The specific structural parameters of the subject's head mainly include head width, head length, head circumference, head sagittal arc, tragus-to-tragus arc, morphological facial height and head height, as shown in FIG. 3(a) to FIG. 3(f).

To more conveniently implement of the irradiation protocol, the reference calvarial region of the subject's head is divided into an anterosuperior cranial region 401a, a cranial vertex region 401b, a left lateral cranial region 401c, a right lateral cranial region 401d, and a posterior cranial region 401e, and the specific division scheme may be adjusted according to actual needs. For example, the division method shown in FIG. 4(a) to FIG. 4(e) may be adopted. Specifically, this division method is based on the international 10-10 standard system. The international 10-10 standard system is an electrode placement standard for electroencephalography (EEG) recording, which provides an accurate and consistent way to mark and locate electrodes on the head. That is to say, the 10-10 system can be directly applied on the subject's head without transcranial operation. This system is an extension of the earlier 10-20 system proposed by the International Federation of Clinical Neurophysiology for standardizing electrode positions in EEG recordings.

In the 10-10 system, the electrode positions are determined based on anatomical landmarks of the head, including the nasion, inion, and left and right pre-auricular points. Through these landmark points, the anterior-posterior and left-right midlines of the head can be determined, and then electrodes are placed at a ratio of 10%.

The naming convention of the 10-10 system is based on the 10-20 system, but it provides denser electrode placement, especially in the basal and anterior parts of the temporal lobe and the frontal lobe, locating positions that are often neglected in the 10-20 system. In addition, the 10-10 system also introduces some new electrodes to allow for more precise position division at the junctions between brain regions, for example, FC represents electrodes between frontal and central regions, FT represents electrodes between frontal and temporal regions, CP represents electrodes between the central and parietal regions, and PO represents electrodes between parietal and occipital regions. The electrode placement of the 10-10 system can effectively eliminate the influence of individual differences in head shape and dimension, so that the same electrode position can be substantially accurately located to the same brain region node in different individuals. The denser electrode positions, especially for electrode positions of the temporal lobe and frontal lobe, enables a more refined division of the anterosuperior cranial region 401a, cranial vertex region 401b, left lateral cranial region 401c, right lateral cranial region 401d, and posterior cranial region 401e on the surface of the calvarial region (external to the skull, scalp, and hair), and accordingly achieves a more refined division of the brain regions (frontal lobe, parietal lobe, temporal lobe) below the skull and dura mater.

In some embodiments, the anterosuperior cranial region 401a, cranial vertex region 401b, left lateral cranial region 401c, right lateral cranial region 401d, and posterior cranial region 401e may be bounded based on their boundary lines. Referring to FIG. 4(a) to FIG. 4(e), the anterosuperior cranial region 401a is within a first region 402a enclosed by a first boundary line 400a and the total boundary line 400. According to the 10-10 standard system, the first boundary line 400a sequentially passes between the following electrode positions: between F7 and FT7, between F5 and FC5, between FC3 and C3, between FC1 and C1, between FCZ and CZ, between FC2 and C2, between FC4 and C4, between F6 and FC6, and between F8 and FT8. Note that the small protrusions at each electrode position in FIG. 4(a) to FIG. 4(f) are only for clearly presenting the electrode positions in the present application, and such small protrusions may not actually be provided. For example, small protrusions may not be provided on the surface of the reference head phantom, which will not be described in detail herein.

The cranial vertex region 401b is within a second region 402b enclosed by a second boundary line 400b. According to the 10-10 standard system, the second boundary line 400b sequentially passes between the following electrode positions: between FC3 and C3, between FC1 and C1, between FCZ and CZ, between FC2 and C2, between FC4 and C4, between C6 and C4, between CP6 and CP4, between P6 and P4, between PO4 and P4, between PO4 and P2, between POZ and P2, between POZ and PZ, between POZ and P1, between PO3 and P1, between PO3 and P3, between P5 and P3, between CP5 and CP3, and between C5 and C3. The left lateral cranial region 401c is within a third region 402c enclosed by a third boundary line 400c and the total boundary line 400. According to the 10-10 standard system, the third boundary line 400c sequentially passes between the following electrode positions: between FT7 and F7, between FC5 and F5, between FC5 and FC3, between C5 and C3, between CP5 and CP3, between P5 and P3, between P5 and PO5, and between P7 and PO7. The right lateral cranial region 401d is within a fourth region 402d enclosed by a fourth boundary line 400d and the total boundary line 400. According to the 10-10 standard system, the fourth boundary line 400d sequentially passes between the following electrode positions: between FT8 and F8, between FC6 and F6, between FC6 and FC4, between C6 and C4, between CP6 and CP4, between P6 and P4, between P6 and PO6, and between P8 and PO8. The posterior cranial region 401e is within a fifth region 402e enclosed by a fifth boundary line 400e and the total boundary line 400. According to the 10-10 standard system, the fifth boundary line 400e sequentially passes between the following electrode positions: between P7 and PO7, between P5 and PO5, between P3 and PO3, between P1 and POZ, between PZ and POZ, between P2 and POZ, between P4 and PO4, between P6 and PO6, and between P8 and PO8. In the present application, the expression “passes between electrode positions A and B” as used in this application is intended to mean passing through an intermediate point on the line connecting electrode positions A and B. For example, the intermediate point may be a midpoint of the line, or another point on the line, for example, a point where the distance ratio from electrode positions A and B is 1:2. In some embodiments, for the same boundary line, such as the fifth boundary line 400e, the points passing between pairs of electrode positions may be located at different ratios on the connecting line, such that the sequentially connected boundary line is smooth.

In some embodiments, the anterosuperior cranial region 401a, cranial vertex region 401b, left lateral cranial region 401c, right lateral cranial region 401d, and posterior cranial region 401e may also be bounded based on the electrode positions they include. Referring to FIG. 4(a) to FIG. 4(e), the anterosuperior cranial region 401a forms a region including electrode positions FP2, FPZ, FP1, AF3, AF4, AF7, AF8, AFZ, FZ, F1, F2, F3, F4, F5, F6, F7, F8, FC1, FC2, FC3, FC4, and FCZ. The cranial vertex region 401b forms a region including electrode positions CZ, C1, C2, C3, C4, CPZ, CP1, CP2, CP3, CP4, PZ, P1, P2, P3, and P4. The left lateral cranial region 401c forms a region including electrode positions FT7, FC5, T7, C5, TP7, CP5, P7, and P5, and the right lateral cranial region 401d forms a region including electrode positions FT8, FC6, T8, C6, TP8, CP6, P8, and P6. The posterior cranial region 401e forms a region including electrode positions PO7, PO5, PO3, POZ, PO4, PO6, PO8, O1, OZ, and O2.

As an example, in FIG. 4(a) to FIG. 4(e), the anterosuperior cranial region 401a, cranial vertex region 401b, left lateral cranial region 401c, right lateral cranial region 401d, and posterior cranial region 401e are connected to each other without apertures, and occupy the entire surface area of the reference calvarial region 401 of the subject's head, but this is only an example. In some embodiments, the anterosuperior cranial region 401a, cranial vertex region 401b, left lateral cranial region 401c, right lateral cranial region 401d, and posterior cranial region 401e each occupies 70% or more of the surface area of the corresponding first region 402a, second region 402b, third region 402c, fourth region 402d, and fifth region 402e divided by respective boundary lines, that is, an occupancy ratio as low as 70% is also acceptable. Sufficient irradiation delivered to (received by) 70% of the surface area of each region achieves a comprehensive AD inhibition effect throughout the entire brain through the optical diffusion effect of the subcranial brain tissue and the conduction between brain region nodes.

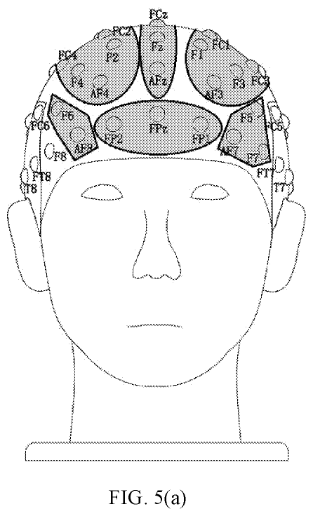

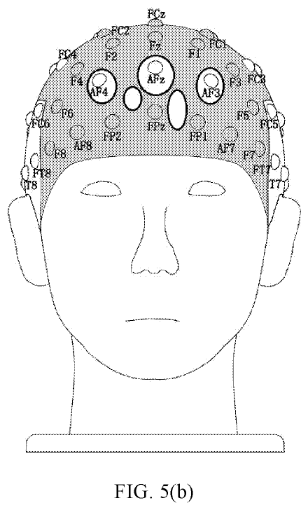

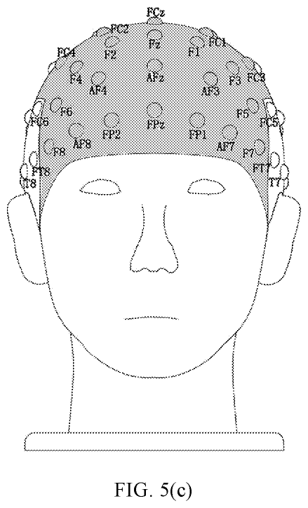

The above division methods are merely examples. Although the division of the anterosuperior cranial region 401a, cranial vertex region 401b, left lateral cranial region 401c, right lateral cranial region 401d, and posterior cranial region 401e may be adjusted as needed, it is preferable that the respective regions mainly correspond to the frontal lobe, parietal lobe, left temporal lobe, right temporal lobe and occipital lobe, respectively. For example, each of the anterosuperior cranial region 401a, cranial vertex region 401b, left lateral cranial region 401c, right lateral cranial region 401d, and posterior cranial region 401e may be divided into multiple island-shaped sub-regions around each group of electrode sites, as shown in FIG. 5(a). For example, each region may also be divided into a single continuous state-shaped region with small holes opened therein, and some small holes may be opened around the electrode sites, as shown in FIG. 5(b).

More preferably, the anterosuperior cranial region 401a is distributed across the prefrontal lobe, frontal lobe, and the region between the frontal lobe and the central region, as shown in FIG. 5(c). Furthermore, the cranial vertex region 401b is distributed across the central region, the region between the central region and the parietal lobe, and parietal lobe; the left lateral cranial region 401c and right lateral cranial region 401d are distributed across the region between the frontal lobe and the temporal lobe, temporal lobe, and the region between the temporal lobe and the parietal lobe; and the posterior cranial region 401e is distributed across the region between the parietal lobe and the occipital lobe, and occipital lobe. More preferably, the left lateral cranial region 401c and the right lateral cranial region 401d are distributed across the lower region between the frontal lobe and the central region, the region between the frontal lobe and the temporal lobe, the temporal lobe, the lower central region, inferior parietal lobe, the region between the temporal lobe and the parietal lobe, and lower region between the central region and the parietal lobe. Through such distribution, each region includes the connection sites between brain regions, and sufficient irradiation is also delivered to the connection sites during irradiation by regions, thereby making the transmission path of the “modulation” and “activation” responses of cell populations smoother.

In some embodiments, during the manufacturing process of the phototherapy apparatus, a reference head phantom of a population of treatment subjects may be used to perform simulation, modeling, or testing of the attenuation and transmission of near-infrared light. Specifically, a reference head phantom with representative dimensions may be selected according to the population of treatment subjects. For example, the concentrated age group for AD patients is over 60 years old, and for the population of treatment subjects in this age group, the parameters of the reference head phantom may be: head width is 140 to 166 mm, head length is 170 to 196 mm, head circumference is 525 to 583 mm, morphological facial height is 104 to 130 mm, head sagittal arc is 304 to 372 mm, tragus-to-tragus arc is 320 to 375 mm, head height is 206 to 253 mm.

Specifically, the parameter range of this reference head phantom falls within the intersection of the distribution ranges of the P1, P5, P10, P50, P90, P95 and P99 parameter values for females in this age group and the P1, P10, P50, P90, P95 and P99 parameter values for males in this age group, so it has good representativeness for both males and females in this age group.

In some embodiments, the parameters of the reference head phantom may be refined as: head width is 152 mm, head length is 184 mm, head circumference is 536.7 mm, morphological facial height is 109.3 mm, head sagittal arc is 355.6 mm, tragus-to-tragus arc is 324.1 mm, and head height is 206 mm. At least some of the refined parameters are determined based on the P50 parameter values of females and males in this age group. For example, the head width and head length here are the average values of the two corresponding P50 parameter values. The P50 parameter values for females in this age group are as follows: head width is 149 mm, head length is 180 mm, head circumference is 548 mm, morphological facial height is 111 mm, head sagittal arc is 335 mm, tragus-to-tragus arc is 342 mm, head height is 228 mm. The P50 parameter values for males in this age group are as follows: head width is 155 mm, head length is 188 mm, head circumference is 565 mm, morphological facial height is 121 mm, head sagittal arc is 343 mm, tragus-to-tragus arc is 351 mm, head height is 231 mm. It can be seen that the parameters of the reference head phantom are in good agreement with the P50 parameters of both females and males in this age group, thus achieving better representativeness. Furthermore, the cephalo-facial index of this parameter is 82%, which is also consistent with the cephalo-facial index range of brachycephaly, the dominant head type among Chinese (and even East Asian populations). Therefore, the parameters of this reference head phantom have particularly good representativeness among Chinese and East Asian populations. In some embodiments, for populations with other cephalo-facial indexes, such as but not limited to the major human populations in Europe, South Asia, and Africa, each parameter can be adaptively adjusted to have good representativeness.

The electrode positions of the international 10-10 standard system may be measured and marked on the reference head phantom. In some embodiments, by adopting the division of the anterosuperior cranial region 401a, cranial vertex region 401b, left lateral cranial region 401c, right lateral cranial region 401d, and posterior cranial region 401e as described above, simulation, modeling, or testing may be performed according to the details of the locally focused stimulation protocol or multi-region balanced stimulation protocol intended to be implemented. In some embodiments, according to the simulation or modeling results, it may be verified whether the designed phototherapy apparatus can satisfy the required coordinated irradiation condition under the intended implementation protocol. The configuration of the phototherapy apparatus, especially the three-dimensional spatial arrangement of the irradiation panels, can be adjusted accordingly. After the verification is completed, a prototype of the designed phototherapy apparatus may be manufactured, and a reference head phantom can be irradiated with the prototype for testing and verification. It can be understood that if the actual test results on the reference head phantom are good, given the good representativeness of the reference head phantom dimensions among the individual head dimensions of the population of treatment subjects, the subsequent phototherapy intervention on the population of treatment subjects will exhibit a highly consistent coordinated irradiation condition. Such a manufacturing process can balance manufacturing cost and therapeutic effect.

Specifically, in some embodiments, the coordinated irradiation condition of the irradiated surface area ratio and the delivered radiant power level may be defined as follows. In case that the irradiated surface area ratio of the near-infrared light delivered to the subject's head is from 30% to 40%, the spatiotemporally averaged irradiance over (i.e., landing on and received by) the irradiated surface area of the subject's head is 117 mW/cm² or more and 230 mW/cm² or less. For example, under this irradiated surface area ratio, the numerical value of the spatiotemporally averaged irradiance (in mW/cm²) over the irradiated surface of the subject's head may be any one of all numerical points within the numerical range of 117 to 230, starting from 117 and distributed at intervals of 0.5-1.

In case that the irradiated surface area ratio is between 40% and 65%, the spatiotemporally averaged irradiance over (i.e., irradiated onto) the irradiated surface area of the subject's head is 110 mW/cm² or more and 230 mW/cm² or less. For example, under this irradiated surface area ratio, the numerical value of the spatiotemporally averaged irradiance (in mW/cm²) over the irradiated surface of the subject's head may be any one of all numerical points in the numerical range of 110 to 230, starting from 110 and distributed at intervals of 0.5-1.

In case that the irradiated surface area ratio is 65% or more, the average coordinated energy delivery to the subject's head ranges from 2750 W·% to 14100 W·%, wherein the average coordinated energy delivery is a product of the irradiated surface area ratio (its percentage value) and the average total radiant power incident upon the head, and the spatiotemporally averaged irradiance over the irradiated surface of the subject's head is 230 mW/cm² or less. For example, under this irradiated surface area ratio, the numerical value of the average coordinated energy delivery (in W·%) to the subject's head may be any one of all numerical points in the numerical range of 2750 to 14100, starting from 2750 and distributed at intervals of 10.

However, in the actual manufacturing and verification process, it is not necessary to exhaust each of the above irradiated surface area ratios. Selecting part of them can also simulate, model, or test whether the phototherapy apparatus satisfies the corresponding coordinated irradiation condition.

By adopting the above flexible combinations of the anterosuperior cranial region 401a, cranial vertex region 401b, left lateral cranial region 401c, right lateral cranial region 401d, and posterior cranial region 401e, multiple representative irradiated surface area ratios can be obtained, so as to conveniently realize the simulation and verification of the locally focused stimulation protocol or the multi-region balanced stimulation protocol accordingly.

As an example, as shown in FIG. 4(a) to FIG. 4(e), taking the first region 402a, the second region 402b, the third region 402c, the fourth region 402d and the fifth region 402e as the anterosuperior cranial region 401a, the cranial vertex region 401b, the left lateral cranial region 401c, the right lateral cranial region 401d and the posterior cranial region 401e respectively, the ratio of the surface area of each region with respect to the surface area of the reference calvarial region is shown in Table 1:

TABLE 1
Ratio of the surface area of the anterosuperior cranial region, cranial vertex
region, left lateral cranial region, right lateral cranial region, and posterior cranial region
with respect to that of the reference calvarial region

| | | | | Right | |
|---|---|---|---|---|---|
| | | Cranial | Left Lateral | Lateral | Posterior |
| Region | Anterosuperior | vertex | cranial | cranial | cranial |
| Name | cranial region | region | region | region | region |
| Surface | 35.7% | 23.51% | 14.17% | 14.17% | 12.45% |
| Area Ratio | | | | | |

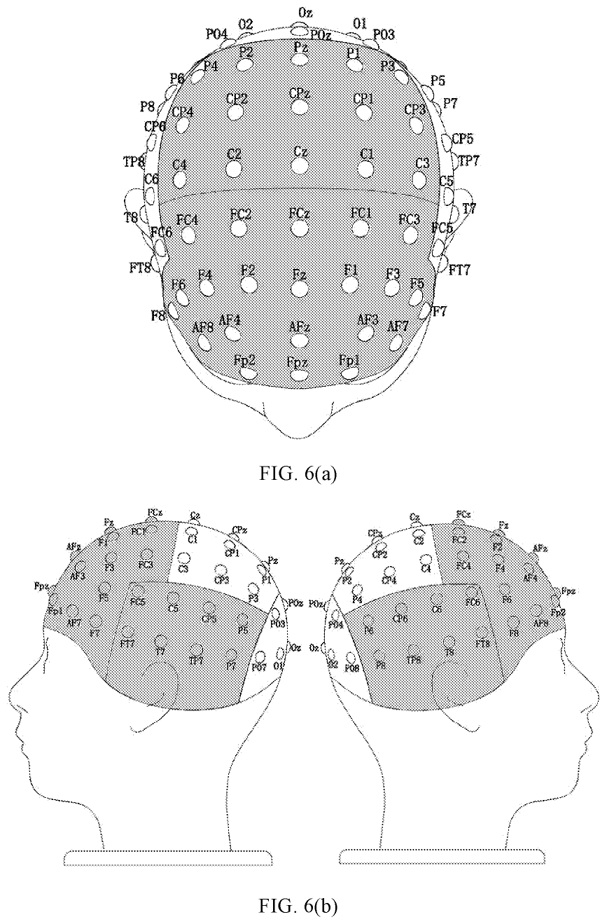

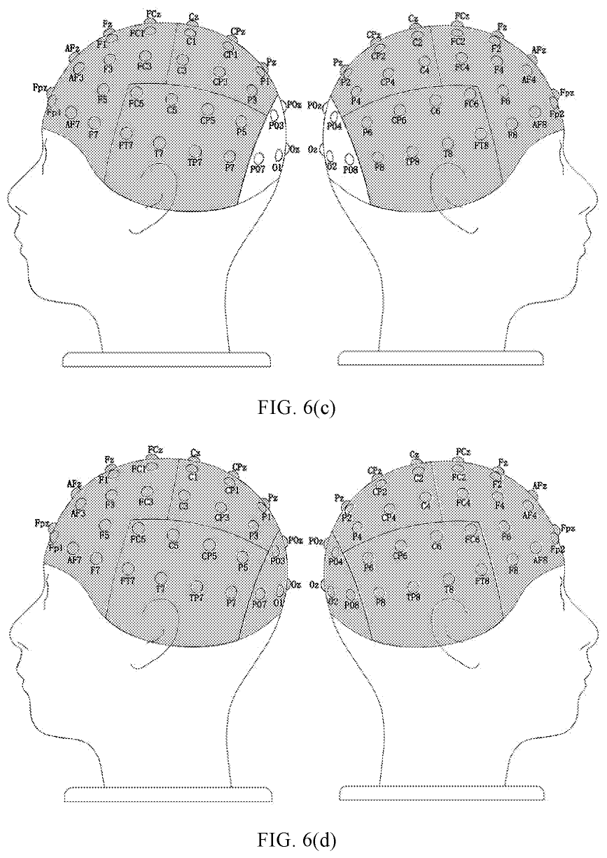

That is to say, the near-infrared light irradiation may be delivered to any one of the following regions of the subject's head: the anterosuperior cranial region; the anterosuperior cranial region and the cranial vertex region, as shown in FIG. 6(a); the anterosuperior cranial region, the left lateral cranial region and the right lateral cranial region, as shown in FIG. 6(b); the anterosuperior cranial region, the left lateral cranial region, the right lateral cranial region, and the cranial vertex region, as shown in FIG. 6(c); and the anterosuperior cranial region, the left lateral cranial region, the right lateral cranial region, the cranial vertex region, and the posterior cranial region, as shown in FIG. 6(d). As an example, when the irradiation is only delivered to the anterosuperior cranial region of the subject's head, the irradiated surface area ratio is 35.7% (satisfying the range between 30% and 40%), and the spatiotemporally averaged irradiance irradiated over the irradiated surface of the subject's head is 117 mW/cm² or more and 230 mW/cm² or less. When the irradiation is delivered only to the anterosuperior cranial region and the cranial vertex region of the subject's head, the irradiated surface area ratio is 59.21% (satisfying the range between 40% and 65%), or when the irradiation is only delivered to the anterosuperior cranial region, the left lateral cranial region and the right lateral cranial region of the subject's head, the irradiated surface area ratio is 64.03% (satisfying the range between 40% and 65%), and the spatiotemporally averaged irradiance over the irradiated surface of the subject's head is 110 mW/cm² or more and 230 mW/cm² or less. When the irradiation is delivered to the anterosuperior cranial region, the left lateral cranial region, the right lateral cranial region, and the cranial vertex region of the subject's head, the irradiated surface area ratio is 87.54%, and the average coordinated energy delivery ranges from 2750 W·% to 10800 W·% of the surface area of the reference calvarial region. When the irradiation is delivered to the anterosuperior cranial region, the left lateral cranial region, the right lateral cranial region, the cranial vertex region, and the posterior cranial region of the subject's head, the irradiated surface area ratio is 100%, and the average coordinated energy delivery ranges from 2750 W·% (i.e., % of the surface area of the reference calvarial region) to 14100 W·% (i.e., % of the surface area of the reference calvarial region). That is to say, after the reference head phantom of the subject's head is manufactured, if the regions are divided as aforementioned, as long as targeted irradiation is performed according to the corresponding combination manner of the above regions, multiple levels of irradiated surface area ratios from 30% to 100% may be conveniently applied, such as irradiated surface area ratio of 35.7%, 59.21%, 64.03%, 87.54%, or 100%. In some embodiments, any irradiated surface area ratio within the range of 30% to 100% can also be flexibly achieved by irradiating only part of each region or through combining irradiation of several regions, for example, any one of the percentage values starting from 30% and distributed at intervals of 5%.

Correspondingly, the phototherapy apparatus can be adjusted, and the irradiation parameters under a corresponding combination manner can be detected to check whether the spatiotemporally averaged irradiance over the irradiated surface of the subject's head or the average coordinated energy delivery to the subject's head satisfies the intended coordinated irradiation condition. If so, the calibration of the irradiation parameters of the phototherapy apparatus under this combination mode is completed. Furthermore, after leaving the factory, if the user adopts a customed combination mode of the regions, the corresponding calibration can also be performed as above, so to ensure that the desired coordinated irradiation condition is satisfied under the customed combination mode of the regions, thereby ensuring the therapeutic effect for AD.

In some embodiments, the spatiotemporally averaged irradiance over the irradiated surface (i.e., the first proportion of the surface area of the reference calvarial region of the subject's head) when the near-infrared light irradiation is delivered to a first proportion of the surface area of the reference calvarial region of the subject's head is greater than the spatiotemporally averaged irradiance over the irradiated surface (i.e., the second proportion of the surface area of the reference calvarial region of the subject's head) when the near-infrared light irradiation is delivered to a second proportion of the surface area of the reference calvarial region of the subject's head, wherein the first proportion is less than the second proportion. The first proportion and the second proportion are selected from any one of the following. For example, both the first proportion and the second proportion may be from 30% to 40%. For example, the first proportion may be from 30% to 40% and the second proportion may be between 40% and 65%. For example, both the first proportion and the second proportion may be between 40% and 65%. For example, the first proportion may be between 40% and 65% and the second proportion may be 65% or more. For another example, both the first proportion and the second proportion may be 65% or more. In some embodiments, by means of performing simulation modeling of the light irradiation process of the phototherapy apparatus with respect to the subject's head, the desired spatiotemporally averaged irradiance corresponding to various irradiated ratios of surface area may be analyzed. Specifically, considering the energy attenuation of near-infrared light with corresponding wavelength in each part of the scalp, each part of the skull, cerebrospinal fluid, dura mater, arachnoid mater, pia mater, etc., simulation can be performed with the purpose for achieving an irradiated surface area ratio and spatiotemporally averaged irradiance over the target brain region such as the prefrontal lobe, so as to determine the irradiated region on the surface of the reference calvarial region of the subject's head and the corresponding spatiotemporally averaged irradiance (i.e., over the irradiated region). During the simulation, the irradiated surface area ratio can be adjusted by adjusting the irradiated region(s) on the surface of the reference calvarial region of the subject's head, and the spatiotemporally averaged irradiance over the irradiated surface of the reference calvarial region of the subject's head can be adjusted accordingly based on the above relationship between the spatiotemporally averaged irradiances for the first proportion and the second proportion, so as to obtain a specific coordinated irradiation protocol for the surface of the reference calvarial region of the subject's head, thereby facilitating subsequent calibration, verification, and actual operation of the phototherapy apparatus.

As described above, the inventors have creatively found that for the therapeutic effect of phototherapy in treating AD, the time-averaged radiant power level delivered to the brain tissue and the irradiated surface area ratio are both important factors for coordinated action. Accordingly, the coordinated irradiation condition, which needs to be satisfied by the near-infrared light irradiation delivered to the subject's head, can characterize the important factor-the time-averaged (delivered) radiant power level in various ways. For example, the time-averaged radiant power level may be characterized as a spatiotemporally averaged irradiance, or as a total radiant power, or as an irradiance or radiant power based composite parameter (such as product, integral, etc.) calculated with the irradiated surface area. In some embodiments, the average total irradiation power of the near-infrared light irradiation delivered to the subject's head is 23 to 140 W, or 29 to 120 W, or 31 to 100 W. In some embodiments, a corresponding average total irradiation power may also be adapted according to the course of AD.

For AD patients with unclear or discretely distributed target regions, the multi-region balanced stimulation protocol is preferable. For example, the irradiation may be delivered to the anterosuperior cranial region, the left lateral cranial region, the right lateral cranial region, and the cranial vertex region, or the irradiation may be delivered to the anterosuperior cranial region, the left lateral cranial region, the right lateral cranial region, the cranial vertex region, and the posterior cranial region. The division for each region can refer to the description in any embodiments, but is not limited thereto. Wherein, the spatiotemporally averaged irradiance over the irradiated surface of (i.e., irradiated onto) the anterosuperior cranial region is 50 to 120 mW/cm², the spatiotemporally averaged irradiance over the irradiated surface of the cranial vertex region is 60 to 120 mW/cm², and the spatiotemporally averaged irradiance over the irradiated surface of the left lateral cranial region and right lateral cranial region is 32 to 85 mW/cm². In some embodiments, the spatiotemporally averaged irradiance over the irradiated surface of the anterosuperior cranial region, cranial vertex region, left lateral cranial region, or right lateral cranial region may be any values obtained from the above corresponding ranges starting from the lower limit at intervals of 4-10 mW/cm².

In some embodiments, if the anterosuperior cranial region 401a, the left lateral cranial region 401c, the right lateral cranial region 401d, the cranial vertex region 401b and the posterior cranial region 401e respectively include the corresponding electrode positions as shown in FIG. 4(a) to FIG. 4(e), the requirements for the spatiotemporally averaged irradiance may be refined to the clusters of electrode sites (i.e., electrode positions), so as to accurately reflect the fluctuation of the time-averaged irradiance, especially at the electrode sites at the region boundaries.

In some embodiments, in case that the irradiation is delivered to at least the anterosuperior cranial region, the left lateral cranial region, the right lateral cranial region, and the cranial vertex region, the time-averaged irradiance at (i.e., irradiated onto) electrode positions Fp1, Fpz, Fp2, AF3, AFz, AF4, F5, F3, F1, Fz, F2, F4, F6, FC1, FC2, FC3, FC4, FC5, FC6, C1, C2, C3, Cz, C4, C5, C6, CP1, CP2, CPz, CP3, CP4, P1, P2, and Pz is 50-90 mW/cm². The time-averaged irradiance at (i.e., irradiated onto) electrode positions FCz, P3, P4, TP7, FT7, T7, FT8, T8, POZ, P6, TP8, CP6, P5, CP5, F7, F8, AF7, and AF8 is 25-65 mW/cm².

In some embodiments, in case that the irradiation is delivered to the anterosuperior cranial region, the left lateral cranial region, the right lateral cranial region, cranial vertex region, and posterior cranial region, the time-averaged irradiance at electrode positions P8, PO8, O1, O2, Oz, P7, and PO7 is 1 mW/cm² to 30 mW/cm², that is, it can fluctuate within this range.

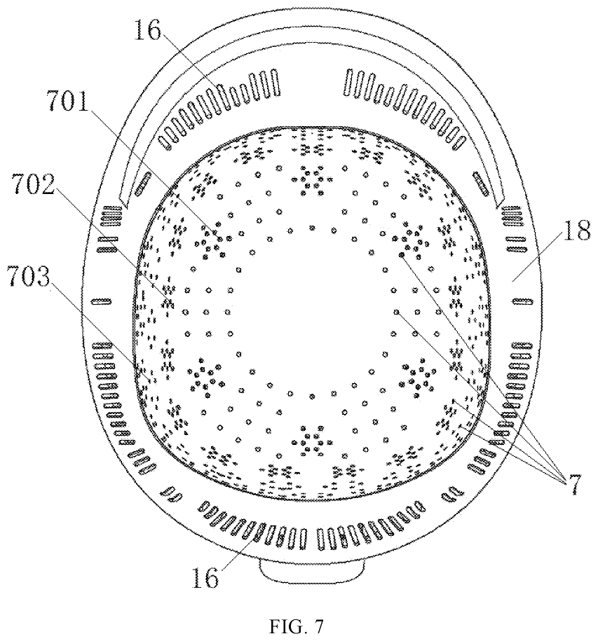

For the multi-region balanced stimulation protocol, the head-worn device 100 with the loose headgear design shown in FIG. 1(b) may be adopted. In some embodiments, adjacent near-infrared irradiation units (e.g., LED light-emitting units) have a predetermined spacing therebetween, and each near-infrared irradiation unit has a predetermined emission angle, such that, when the subject's head is positioned in place within the accommodation space, near-infrared light is emergent from each position on a peripheral irradiation curved surface corresponding to a reference calvarial region of the subject's head. The phototherapy apparatus and its head-worn device 100 will be further described below with reference to FIG. 7 to FIG. 9, wherein the peripheral irradiation curved surface is implemented as the irradiation surface of the transparent inner housing 3, and the surface area of the peripheral irradiation curved surface is 1200 to 1700 cm².

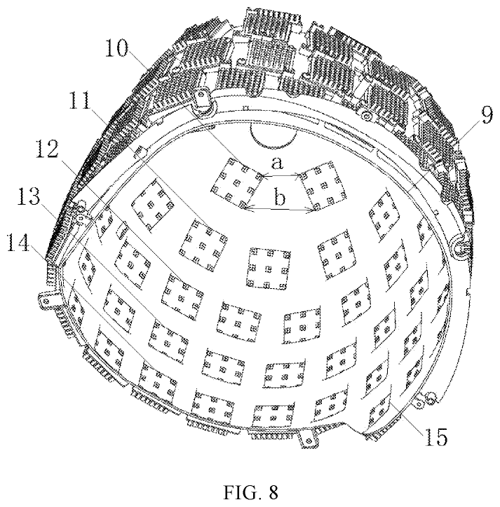

In some embodiments, a plurality of irradiation panels 5 are arranged on the middle housing 2 via an irradiation panel fixing housing 9. The irradiation panel fixing housing 9 is disposed on the side of the middle housing 2 adjacent to the outer housing 1. As shown in FIG. 8, in some embodiments, an irradiation panel fixing housing 9 is disposed within an irradiation panel accommodating cavity 4, the irradiation panel fixing housing 9 is provided on the side of the middle housing 2 adjacent to the outer housing 1. The plurality of irradiation panels 5 are arranged in layers from top to bottom on the irradiation panel fixing housing 9, and the spacing between two irradiation panels 5 on the upper layer is larger than the spacing between two adjacent irradiation panels 5 on the lower layer, so as to ensure an effective phototherapy effect for different brain regions of the patient. In some embodiments, the arrangement density of the irradiation panels corresponding to the patient's top position is relatively low, while denser irradiation panels may be provided at other head positions except for the top position of the patient, but this is only an example. Specifically, the inventors have creatively found that, especially in the head-worn device 100 shown in FIG. 7 to FIG. 9, when the subject's head is positioned in place, a space of at least several centimeters, or even close to 10 centimeters, is reserved at the top of the head to reduce a sense of compression on the subject during phototherapy, but the near-infrared light emitted by a plurality of light-emitting units can overlap on the surface of the subject's head after propagating through the space, and the near-infrared light beams emitted by the respective irradiation panels can also overlap on the surface of the subject's head after propagating through the space, so that the irradiance on the surface of the subject's head can be maintained at a level of, for example, 80-120 mW/cm². Note that the multi-layer circumferential surrounding distribution of the plurality of irradiation panels 5 is only an example of the array of near-infrared irradiation units. The array of near-infrared irradiation units may also adopt LEDs, laser diodes, or optical fibers for transmitting near-infrared light from the outside, etc., which will not be described in detail herein. In some embodiments, the array of near-infrared irradiation units may be specifically configured to emit near-infrared light with a duty cycle of 30% to 70%, a wavelength of 650 to 1100 nm, and a frequency falling within the Alpha wave frequency range, the Gamma wave frequency range, or a frequency range adjacent thereto. Wherein, each individual near-infrared irradiation device constituting the near-infrared irradiation unit may be an LED with an average radiant power of 90 mW or more.

In other embodiments of the present application, the plurality of irradiation panels 5 may be directly arranged on the middle housing 2.

In some embodiments, as shown in FIG. 8, six layers of irradiation panels 5 are arranged on the irradiation panel fixing housing 9 at intervals, and the irradiation panels 5 include at least one of the following arrangement modes.

Mode 1: In the first layer of irradiation panels 10 closest to the top of the headgear, there is a first gap between two adjacent irradiation panels, the length of the narrowest position a of the first gap ranges from 23 mm to 26 mm, and the length of the widest position b of the first gap ranges from 57 mm to 60 mm. Optionally, the length of the narrowest position a of the first gap ranges from 23.5 mm to 25.5 mm, and the length of the widest position b of the first gap ranges from 57.5 mm to 59.5 mm. Preferably, the length of the narrowest position a of the first gap is about 24 mm, and the length of the widest position b of the first gap is about 59 mm. Note that in the present application, reference to “about” a certain value is intended to take into account the measurement error.

Mode 2: In the second layer of irradiation panels 11 adjacent to the first layer of irradiation panels 10, there is a second gap between two adjacent irradiation panels, the length of the narrowest position of the second gap ranges from 15 mm to 18 mm, and the length of the widest position of the second gap ranges from 41 mm to 44 mm. Optionally, the length of the narrowest position of the second gap ranges from 16 mm to 17.5 mm, and the length of the widest position of the second gap ranges from 41 mm to 43 mm. Preferably, the length of the narrowest position of the second gap is about 16.7 mm, and the length of the widest position of the first gap is about 42.5 mm.

Mode 3: In the third layer of irradiation panels 12 located below the second layer of irradiation panels 11, there is a third gap between two adjacent irradiation panels, the length of the narrowest position of the third gap ranges from 13 mm to 16 mm, and the length of the widest position of the third gap ranges from 24 mm to 27 mm. Optionally, the length of the narrowest position of the third gap ranges from 13.5 mm to 15.5 mm, and the length of the widest position of the third gap ranges from 24 mm to 26 mm. Preferably, the length of the narrowest position of the third gap is about 14.2 mm, and the length of the widest position of the third gap is about 25.3 mm.

Mode 4: In the fourth layer of irradiation panels 13, the fifth layer of irradiation panels 14 and the sixth layer of irradiation panels 15, which are located below the third layer of irradiation panels 12 and arranged sequentially from top to bottom, there is a fourth gap between two adjacent irradiation panels in the fourth layer of irradiation panels 13, the length of the narrowest position of the fourth gap ranges from 12 mm to 15 mm, and the length of the widest position of the fourth gap ranges from 19 mm to 22 mm. Optionally, the length of the narrowest position of the fourth gap ranges from 13.5 mm to 14.5 mm, and the length of the widest position of the fourth gap ranges from 19.5 mm to 21.5 mm. Preferably, the length of the narrowest position of the fourth gap is about 14 mm, and the length of the widest position of the fourth gap is about 20.5 mm.

Mode 5: There is a fifth gap between two adjacent irradiation panels in the fifth layer of irradiation panels 14, the length of the narrowest position of the fifth gap ranges from 12 mm to 15 mm, and the length of the widest position of the fifth gap ranges from 16 mm to 19 mm. Optionally, the length of the narrowest position of the fifth gap ranges from 13 mm to 14.5 mm, and the length of the widest position of the fifth gap ranges from 16.5 mm to 18.5 mm. Preferably, the length of the narrowest position of the fifth gap is about 13.8 mm, and the length of the widest position of the fifth gap is about 18 mm.

Mode 6: There is a sixth gap between two adjacent irradiation panels in the sixth layer of irradiation panels 15, the length of the narrowest position of the sixth gap ranges from 11 mm to 14 mm, and the length of the widest position of the sixth gap ranges from 15 mm to 18 mm. Optionally, the length of the narrowest position of the sixth gap ranges from 12 mm to 14 mm, and the length of the widest position of the sixth gap ranges from 15 mm to 17 mm. Preferably, the length of the narrowest position of the sixth gap is about 13 mm, and the length of the widest position of the sixth gap is about 16.2 mm.

The arrangement of the above structure allows the overall temperature inside the headgear to achieve a dynamic balance on the premise that the irradiation panels 5 irradiate to achieve the phototherapy effect. The setting of the spacing between the irradiation panels 5 and the setting between adjacent layers of the irradiation panels 5 avoid concentrated heat dissipation of the irradiation panels 5, prevent excessive local temperature in the headgear, and save energy consumption.

In an alternative embodiment, as shown in FIG. 8, the gap between two adjacent irradiation panels 5 in each layer of irradiation panels 5 has a narrowest position and a widest position respectively, and the width of the gap between two adjacent irradiation panels 5 on the upper layer is larger than the width of the gap between two adjacent irradiation panels 5 on the lower layer. In this way, on the premise that the irradiation panels 5 are ensured to irradiate to achieve the phototherapy effect, the situation of excessive local temperature inside the phototherapy headgear caused by the dense arrangement of the irradiation panels 5 is significantly reduced. Wherein, due to the structural design of the headgear of the phototherapy apparatus, the inner diameter of the accommodation space tends to gradually increase from top to bottom, so the arrangement spacing in the first layer of irradiation panels 10 becomes wider than that in the second layer of irradiation panels 11. On the premise of ensuring the uniformity of irradiance, this also prevents the heat dissipated by the irradiation panels 5 from accumulating in the headgear, facilitating cooling. In order to achieve the uniformity of irradiance throughout the accommodation space, the first layer of irradiation panels 10 and the second layer of irradiation panels 11 may each has an optimal average radiant power of 75 mW to 125 mW, preferably 90-100 mW.

As shown in FIG. 8, in some embodiments, the vertical spacing between the first layer of irradiation panels 10 and the second layer of irradiation panels 11 is 19 mm to 25 mm, preferably 19.5 mm to 24.5 mm. The vertical spacing between the second layer of irradiation panels 11 and the third layer of irradiation panels 12 is 14 mm to 21 mm, preferably 15 mm to 20 mm.

As shown in FIG. 8, in some embodiments, the vertical spacing between the remaining two adjacent layers of irradiation panels is 13 mm to 19 mm, preferably 14 mm to 18 mm. The setting of vertical spacing between the two adjacent layers of irradiation panels can not only satisfy the irradiation to the entire brain region, but also prevent the heat dissipated by the irradiation panels 5 from accumulating in the accommodation space.

Therefore, the arrangement of the irradiation panels 5 can ensure the irradiation to be delivered to all regions of the patient's head, including the temporal lobe, occipital lobe, frontal lobe and parietal lobe, without affecting the normal progress of phototherapy, and can also improve the comfort during the therapy process.

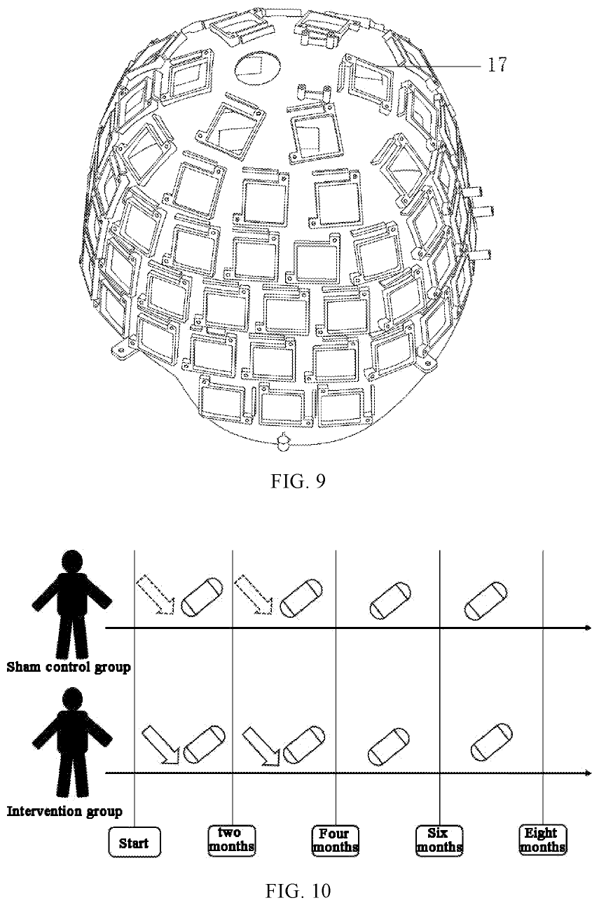

In addition, as shown in FIG. 9, the irradiation panels 5 are connected to irradiation panel fixing frames 17, and the irradiation panels 5 are fixed on the irradiation panel fixing housing 9 via the irradiation panel fixing frames 17. In some embodiments, the spacing between adjacent irradiation panel fixing frames 17 in each layer of irradiation panel fixing frames 17 is similar to or the same as the spacing between corresponding adjacent irradiation panels 5.

Returning to FIG. 7 in conjunction with FIG. 1(b), in some embodiments, the headgear is provided with an annular connecting portion 18, and the bottoms of the outer housing 1 and the middle housing 2 are respectively connected to the connecting portion 18. The connecting portion 18 is provided with a plurality of ventilation openings 16 communicating with the irradiation panel accommodating cavity 4, and the area of each ventilation opening 16 located at the rear end of the headgear is larger than the area of each ventilation opening 16 located at the front end of the headgear. Further, the ventilation openings 16 may be elongated. This can increase the air flow entering the rear end of the headgear to improve the cooling effect of the irradiation panel accommodating cavity 4, and balance the cooling effect of the headgear at the afterbrain and the forehead position. It can also reduce the heat transfer from the irradiation panel accommodating cavity 4 to the cooled gas cavity 6, thereby facilitating an improved cooling effect on the accommodation space.

In some embodiments, the top of the headgear is provided with a gas extraction port, which communicates with the irradiation panel accommodating cavity 4 for extracting hot gas from the irradiation panel accommodating cavity 4.

In some embodiments, a total of 80 irradiation panels 5 are distributed inside the headgear, the size of the near-infrared light emergent port of each irradiation panel 5 is 28 mm×28 mm with a tolerance of ±0.2 mm, and the irradiation units are arranged in a 3×3 array.

We conducted a series of clinical trials using the phototherapy apparatus with this structure. As an example, the near-infrared light emitted by the array of near-infrared irradiation units has a central wavelength of 810 nm, a duty cycle of 50%, and a frequency of 10 Hz, but it should be noted that this is only an example.

In some embodiments, near-infrared light can be emitted with a duty cycle of 30%-70%, a wavelength of 650-1100 nm, and a frequency falling within the Alpha wave frequency range, the Gamma wave frequency range, or a frequency range adjacent thereto, which will not be described in detail herein.

In some embodiments, the array of near-infrared irradiation units is specifically configured to deliver the irradiation of at least 8260 to 42250 Joules to the subject's head during a continuous irradiation period of 5 minutes, as a unit energy delivery. The energy irradiation of 8260 Joules within 5 minutes corresponds to continuous irradiation delivered to the calvarial region of the subject's head with a time-averaged irradiation power of 27.5 W, and when calculated with the surface area of the calvarial region of the subject's head of 612.35 cm², the spatiotemporally averaged irradiance is 45 mW/cm²; the energy irradiation of 42250 Joules within 5 minutes corresponds to continuous irradiation delivered to the calvarial region of the subject's head with a time-averaged irradiation power of 140 W, and when calculated with the surface area of the calvarial region of the subject's head of 612.35 cm², the spatiotemporally averaged irradiance is 230 mW/cm². The inventors found in clinical trials that for some AD patients at the moderate and severe dementia stages, it is difficult to continuously perform treatment without interruption for at least ten minutes to half an hour. Some AD patients have severely impaired cognition and are uncooperative, and some AD patients also have frequent abnormal limb activities, such as muscle stiffness and flexion, amyotrophy and weakness, apraxia, etc. Taking 5 minutes as a unit energy delivery means that each 5 minutes of irradiation is effective in inhibiting AD to a certain extent.

For example, the required energy delivery to the subject's head within a single session of one hour, such as 3-unit energy deliveries, i.e., at least 24800 Joules to 126800 Joules of energy, can be separately administered in separate or continuous effective unit energy deliveries. The single-session cumulative energy delivery in this way can achieve an AD inhibitory effect comparable to that of a continuous single-session cumulative energy delivery for 15 minutes without interruption. In the actual treatment process, even if the treatment is interrupted due to the patient's own reasons or operational failures, the operator only needs to ensure that the cumulative irradiation time within a single session meets the requirements, none of the following is needed: restarting the phototherapy apparatus, or resetting the irradiation energy delivery of the current session, or forcibly interrupting the irradiation. Specifically, if the subject has high compliance and the treatment goes smoothly, he/she may be directly irradiated continuously for 15 minutes. If the subject has low compliance or the treatment does not proceed smoothly, for example, the subject must go to the bathroom after 6 minutes of irradiation, the treatment can be temporarily interrupted and the irradiation can be resumed after the subject returns. The interruption time may be flexibly adjusted according to the subject's needs, as long as the irradiation duration is achieved within one hour. This significantly reduces the difficulty of phototherapy operation for AD patients who are non-compliant due to relatively severe cognitive impairment or motor abnormalities, as well as for a subject population with varying severities of AD (such as the nursing home population across age groups), and even for nursing staff with insufficient experience in caring for AD patients.

In some embodiments, the array of near-infrared irradiation units is specifically configured to deliver the irradiation of at least 24800 Joules to 1014050 Joules to the subject's head within a single day, as a daily cumulative energy delivery. It can be seen that the daily cumulative energy delivery can have a span of nearly 40 times, with its lower limit being 15 minutes of cumulative irradiation at a time-averaged irradiation power of 27.5 W, and its upper limit being 2 hours of irradiation at a time-averaged irradiation power of 140 W. The inventors found that with the phototherapy apparatus of the present application, the daily cumulative energy delivery may be adjusted in a wide range according to the adaptability of the subject. Specifically, if the subject has high compliance with phototherapy and good individual response, the array of near-infrared irradiation units is specifically configured to administer the single-session cumulative energy delivery by continuously irradiating for at least 30 minutes within a single session of one hour, and the single-session cumulative energy delivery can be administered up to four times within a single day to provide an accelerated daily cumulative energy delivery. This accelerated daily cumulative energy delivery corresponds to several days' worth of the lower limit of the daily cumulative energy delivery. In the case that the treatment needs to be interrupted for several days due to specific circumstances of the subject, the accelerated daily cumulative energy delivery can be administered first, thereby increasing the flexibility and convenience of phototherapy. In addition, although the action mechanism is not yet clear, after the accelerated daily cumulative energy delivery is administered to individual volunteers, the improvement in their cognitive performance is even superior to the effect achieved by administering the same cumulative energy delivery over several days. In some embodiments, the weekly cumulative energy delivery to the subject's head can be as low as 24800 Joules, with an irradiation time as short as 15 minutes.

In some embodiments, the array of near-infrared irradiation units is specifically configured to deliver the irradiation of at least 124000 Joules (27.5 W-15 minutes-5 times) to 7100000 Joules (230 mW/cm²-2 hours per day-7 days) to the subject's head within one week in separate single-session cumulative energy deliveries, as a weekly cumulative energy delivery. Specifically, on each day of the week for administering irradiation, the single-session cumulative energy delivery can be administered for 1, 2, 3, 4, 5, 6, 7, or 8 times in separate sessions.

Specifically, the array of near-infrared irradiation units is specifically configured to administer energy delivery at least 8 times as much as the weekly cumulative energy delivery (i.e., weekly cumulative energy delivery*8) within 8 weeks, preferably energy delivery at least 16 times as much as the weekly cumulative energy delivery (i.e., weekly cumulative energy delivery*16) within 16 weeks, as the cumulative energy delivery in a treatment course.

In some embodiments, the treatment course interruption time between two treatment courses does not exceed half of the duration of the treatment course, so as to avoid the deterioration and regression of AD pathological conditions caused by treatment interruption as much as possible.

The applicant conducted clinical trials on the intervention group (i.e., the treatment group) using the phototherapy apparatus shown in FIG. 1(a) and FIG. 1(b). Table 2 shows the spatiotemporally averaged irradiance of the phototherapy apparatus over individual regions of the reference head phantom of the subject's head, as well as the time-averaged irradiance of the irradiation surface of the inner housing 3 at the positions corresponding to the centers of the irradiation panels.

TABLE 2
Spatiotemporally averaged irradiance of the phototherapy apparatus over
individual regions of the reference head phantom

| | Time- | | | | |
|---|---|---|---|---|---|
| | averaged | | | | |
| Time- | irradiance at | | Spatiotempo | | Spatiotempo |
| averaged | the front, | | rally | | rally |
| irradiance at | left, right | | averaged | Total | averaged |
| the upper | and rear | | irradiance at | area | irradiance |
| part of the | parts of the | Total area | each position | ratio of | at each |
| irradiation | irradiation | ratio of the | of the | the | position of |
| surface of the | surface of | anterosuperior | anterosuperior | cranial | the cranial |
| inner | the inner | cranial | cranial | vertex | vertex |
| housing 3 | housing 3 | region | region | region | region |
| 50 to 100 mW/cm² | 100 | 35.7% | 50-120 mW/cm² | 23.51% | 60-120 |
| | mW/cm², | | | | mW/cm² |
| | with an error | | | | |
| | not greater | | | | |
| | than ± 20% | | | | |
| | Spatiotempo | | Spatiotempo | | Spatiotempo |
| | rally | | rally | | rally |
| | averaged | | averaged | Total | averaged |
| | irradiance at | | irradiance at | area | irradiance at |
| Total area | each position | Total area | each position | ratio of | each |
| ratio of the | of the left | ratio of the | of the right | the | position of |
| left lateral | lateral | right lateral | lateral | posterior | the posterior |
| cranial | cranial | cranial | cranial | cranial | cranial |
| region | region | region | region | region | region |
| 14.17% | 32-85 | 14.17% | 32-85 | 12.45% | 1-30 |
| | mW/cm² | | mW/cm² | | mW/cm² |

The applicant conducted a clinical trial on the intervention group (i.e., the treatment group) using the phototherapy apparatus with the operating parameters in Table 3.

TABLE 3
Operating parameters of the phototherapy apparatus

| | Average | | | | | |
|---|---|---|---|---|---|---|
| | Coordinated | | | Time- | | |
| | Energy | | | averaged | | Number of |
| | delivery to the | | Single/ | radiant | Daily | Treatment |
| | Subject's | | Dual/Triple | power of | Treatment | Days per |
| Central Wavelength | Head | Frequency | Wavelength | LED | Protocol | Week |
| 810 nm | 3200 W · % to | 10 Hz | Single | 90-100 | Once, 30 | 6 days |
| | 4800 W · % | | wavelength | mW | minutes | |

In case that the subject's head is positioned in place in the accommodation space formed by the support mechanism, the near-infrared light emitted by the phototherapy apparatus in this clinical trial can cover the anterosuperior cranial region, cranial vertex region, left lateral cranial region, right lateral cranial region and posterior cranial region of the subject's head, with an irradiated surface area ratio of the subject's head of 65% or more, which satisfies the coordinated irradiation condition of the irradiated surface area ratio and the delivered radiant power level. As shown in Table 2 and Table 3, the average coordinated energy delivery to the subject's head is about 3200 W·% to 4800 W·%, and the spatiotemporally averaged irradiance over the irradiated surface of the subject's head is 230 mW/cm² or less.

The inclusion criteria for this clinical trial are as follows: (1) the core clinical criteria for probable Alzheimer's disease (AD) defined by the National Institute on Aging and Alzheimer's Association (NIA-AA) is satisfied; (2) Brain MRI examination (within 6 months) results support the a diagnosis of probable AD; (3) Aged 50-85 years old, regardless of gender; (4) MMSE score <26 points, able to cooperate to complete the scale assessment; (5) Patients are not taking medication during the trial; if they are taking psychiatric or cognitive improvement drugs, the dose must be stable for at least 12 weeks before the trial and remain unchanged during the treatment period.

Exclusion criteria: (1) Presence of MRI contraindications, such as metal implants or claustrophobia; (2) Other types of dementia or other psychiatric or neurological diseases, such as depression or Parkinson's disease; (3) History of stroke or epilepsy; (4) Photosensitivity to sunlight or visible light, or increased skin sensitivity in the treatment area; (5) Severe visual or hearing impairment; (6) History of alcohol or drug addiction; (7) Any other conditions unsuitable for participation in this study.

According to the inclusion criteria and the exclusion criteria, a total of 27 patients were enrolled, with 13 in the intervention group and 14 in the sham control group. The intervention group received whole-head near-infrared light stimulation, with a wavelength of 810 nm and a frequency of 10 Hz. Each participant received one 30-minute treatment per day, 6 days per week, for a continuous period of 4 months. The sham treatment group (sham control group) received exactly the same protocol as the near-infrared intervention group, but used a sham treatment headgear. The light emitted by the sham treatment apparatus was visually identical to that of the near-infrared treatment apparatus, and produced sound and a warm feeling on the scalp similar to the near-infrared treatment apparatus during the treatment process, but the light power was very weak and was basically absorbed by the tissue, and cannot achieve the effect of stimulating brain tissue, as shown in FIG. 10.

Scale assessments were performed at the 2nd and 4th months during the treatment period, as well as at 2 months and 4 months after the end of treatment (the 6th and 8th months), as shown in FIG. 10. The assessors, subjects and their caregivers were blinded to the treatment allocation throughout the study until the end of the study. And during the entire study, none of the relevant personnel discussed the treatment allocation. All subjects believed that they were receiving real near-infrared treatment.

In the end, a total of 18 patients (9 in the intervention group and 9 in the sham control group) completed the 4-month treatment and 4-month follow-up assessment, among which 1 patient only completed the MMSE scale assessment and did not complete the ADAS-cog scale assessment in the scale assessment. The ADAS-Cog scale consists of 12 items covering memory, orientation, language, praxis, attention, etc., which can assess the severity of cognitive symptoms of AD and treatment changes, and is often used for evaluating the efficacy of treatment for mild to moderate AD (usually an improvement of 4 points is used as the clinical judgment criterion for drug efficacy). The MMSE scale is the most widely used cognitive screening scale at home and abroad, covering orientation, memory, attention, calculation, language ability and visuospatial ability. Studies on the MMSE have found that in professional institutions such as memory clinics or in community hospitals, the sensitivity and specificity of the MMSE in distinguishing normal elderly individuals from dementia are both over 80%, presenting favorable value for screening dementia.

To explore the sustainability of the efficacy of near-infrared light on AD patients, follow-up visits were conducted on the subjects after the end of treatment. A total of 14 subjects completed the follow-up visit at the 8th month (i.e., 4 months after the end of treatment, including 8 in the intervention group and 6 in the sham control group).

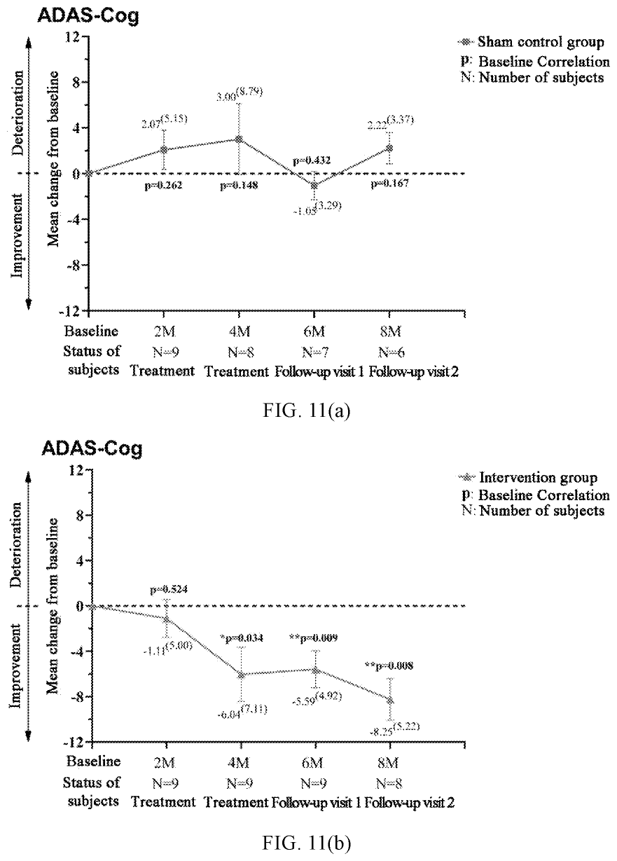

Referring to FIG. 11(a) and FIG. 11(b), it can be seen that after 2 months of phototherapy, the ADAS-cog scale scores of the subjects in the intervention group decreased by an average of 1.11 points from the baseline, and continued to decrease with a steeper slope after 4 months of treatment, with an average decrease of 6.04 points from the baseline, with an intra-group statistical significance of p=0.034<0.05, all of which were significantly superior to the scores of the sham control group. Within 2 months after the end of treatment, the ADAS-cog scale scores of the intervention group exhibited little fluctuation, and still remained at an average decrease of 5.59 points from the baseline within 2 months after the end of treatment, with an intra-group statistical significance of p=0.009<0.05. In the follow-up visit 2 months after the end of treatment, the ADAS-cog scale scores of the intervention group even began to continue to decrease, with an average decrease of 8.25 points from the baseline at 4 months after the end of treatment, with an intra-group statistical significance of p=0.008<0.05. It can be seen from the ADAS-cog scale scores, the phototherapy apparatus of the present application has achieved beneficial effects that have never appeared in papers and related literatures on other existing phototherapy apparatus: during the 4 months of phototherapy, the ADAS-cog scale scores decreased with a steeper slope after the second month compared with the first two months. Two follow-up visits performed at 2-month intervals after the cessation of phototherapy demonstrated that the biochemical reactions caused by the irradiation energy of the near-infrared light delivered to the subject's head continued to trigger the inhibitory effect after the cessation of phototherapy, which not only maintained the inhibitory effect on AD to a certain extent, but also continued to promote the inhibitory effect on AD, without deterioration or regression of the ADAS-cog scale scores.

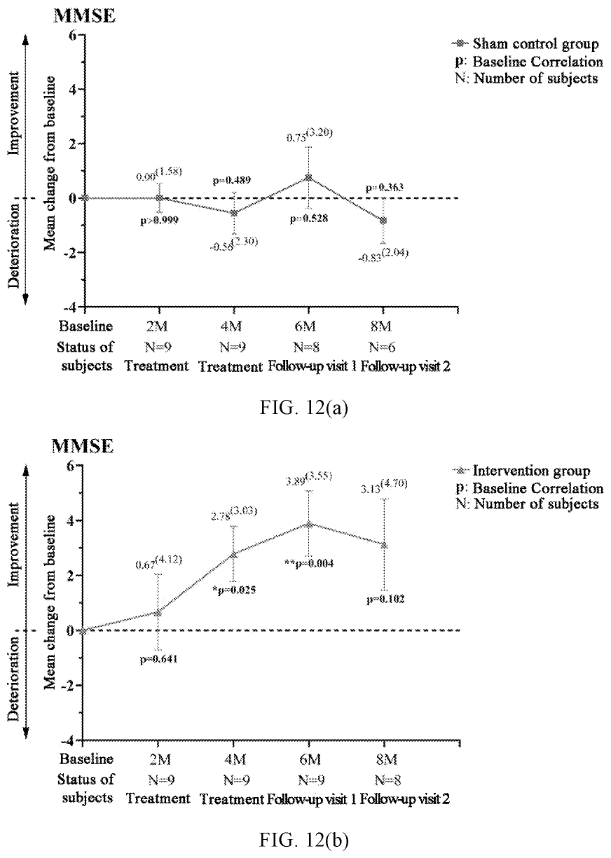

Referring to FIG. 12(a) and FIG. 12(b), it can be seen that after 2 months of phototherapy, the MMSE scale scores of the subjects in the intervention group increased by an average of 0.67 points from the baseline, and during the subsequent 2 months of phototherapy, the MMSE scale scores continued to increase with a steeper slope, reaching an average increase of 2.78 points from the baseline after 4 months of phototherapy, all of which were significantly better than the scores of the sham control group, with an intra-group statistical significance of p=0.025<0.05. The ADAS-cog scale scores and MMSE scale scores in the first 2 months and the last 2 months of phototherapy exhibit similar improvement effects: a continuous improvement with a steeper slope in the last 2 months. Furthermore, similar to the continuous improvement of the ADAS-cog scale scores after the cessation of phototherapy, although the phototherapy has been ended, the MMSE scale scores continued to rise during 2 months after the cessation of phototherapy, even maintaining a rising slope comparable to that during the phototherapy period, reaching an average increase of 3.89 points from the baseline at 2 months after the cessation, with an intra-group statistical significance of p=0.004<0.05. At 4th month after the cessation of phototherapy, the MMSE scale scores of the intervention group remained comparable to those obtained at 4 months of phototherapy, without deterioration or regression. That is to say, from the perspective of MMSE scale scores, the biochemical reactions caused by the near-infrared light energy delivered to the subject's head continue to trigger the inhibitory effect after the cessation of phototherapy, which not only maintains the inhibitory effect on AD to a certain extent, but also continues to promote the inhibitory effect on AD, without deterioration or regression of the scale scores.

Combined with the results of ADAS-Cog scale scores and MMSE scale scores during the phototherapy and in the follow-up visit after the cessation of the phototherapy, the optimized “optical charging” and sustained benefit process of the phototherapy apparatus of the present application mentioned above is also confirmed: the subject's brain has a larger “optical charging capacity”, a deeper “optical charging depth”, and a faster “optical charging speed”. The improvement of scale scores during the first 2 months and the last 2 months of phototherapy is very significant, and occurs with a stable slope without stagnation; the subsequent “sustained effect (i.e., runtime per charge)” capability and sustained benefit are better, the effect of 2 months of phototherapy is maintained at least until 2 months after the cessation of phototherapy, and can even be maintained until at least 4 months after the cessation of phototherapy, and the biochemical reactions caused by “optical charging” continue to trigger the inhibitory effect after the cessation of phototherapy, and continue to promote the inhibitory effect on AD, without deterioration or regression.

Moreover, after receiving 4 months of near-infrared light phototherapy, the resting-state functional magnetic resonance examination results of the subjects in the intervention group showed that the Amplitude of Low-Frequency Fluctuation (ALFF) was enhanced in multiple brain regions including the frontal lobe, occipital lobe and temporal lobe (P<0.05), indicating that the neuronal excitability and spontaneous activity of the subjects were enhanced, providing neuroimaging evidence for the corresponding improvement of cognitive function. At the same time, no adverse events associated with the test instrument were observed in this trial.

Although the phototherapy lasted for 4 months in this clinical trial, the phototherapy showed the characteristics of in-depth regulation, sustained benefits, and even cumulative benefits. Continuous application over a longer period, such as 6 months, 8 months, 10 months . . . more than one year, or even long-term regular use, is expected to produce continuously increased benefits and achieve a more remarkable disease-modifying effect.

Furthermore, although exemplary embodiments have been described herein, scope thereof includes any and all embodiments based on the present disclosure having equivalent elements, modifications, omissions, combinations (e.g., crossed solutions of various embodiments), adaptations, or changes. The elements in the claims will be interpreted broadly based on the language used in the claims, and are not limited to the examples described in the specification or during the implementation of this application, and the examples will be interpreted as non-exclusive. Therefore, the specification and examples are intended to be considered as examples only, and the true scope and spirit are indicated by the following claims and the full scope of their equivalents.

The above description is intended to be illustrative rather than restrictive. For example, the above examples (or one or more aspects thereof) may be used in combination with one another. For example, those skilled in the art may use other embodiments upon reading the above description. In addition, in the above detailed description of the embodiments, various features can be grouped together to simplify the present disclosure. This should not be interpreted as an intention that any unclaimed disclosed feature is essential to any claim. On the contrary, the subject matter of the present disclosure may be less than all the features of a particular disclosed embodiment. Thus, the following claims are hereby incorporated into the detailed description of the embodiments as examples or embodiments, wherein each claim is independently regarded as a separate embodiment, and it is contemplated that these embodiments may be combined with one another in various combinations or arrangements. The scope of the present disclosure shall be determined with reference to the appended claims along with the full scope of equivalents to which such claims are entitled.

The above embodiments are merely exemplary embodiments of the present disclosure, and are not intended to limit the present disclosure. The protection scope of the present disclosure is defined by the claims. Those skilled in the art may make various modifications or equivalent substitutions to the present disclosure within the substance and protection scope of the present disclosure, and such modifications or equivalent substitutions shall also be deemed to fall within the protection scope of the present disclosure.

What is claimed is:

1. A phototherapy apparatus for treating Alzheimer's disease and associated conditions, comprising:
a support mechanism, which is configured to form an accommodation space for a subject's head and to support an array of near-infrared irradiation units; and
the array of near-infrared irradiation units, which are configured to emit near-infrared light into the accommodation space, wherein, when the subject's head is positioned in place within the accommodation space, an irradiated surface area ratio of an irradiated surface area of the near-infrared light irradiation delivered to the subject's head to a surface area of a reference calvarial region of the subject's head is in the range of 30% to 40%, or between 40% and 65%, or 65% or more, a delivered radiant power level of the near-infrared light is provided in coordination with the irradiated surface area ratio within the range, such that the irradiation delivered to the subject's head satisfies a coordinated irradiation condition of the irradiated surface area ratio and the delivered radiant power level, the surface area of the reference calvarial region is the external surface area of a surface of the subject's head within a total boundary line, the total boundary line extends from a Glabella point of the subject's head along supraorbital ridges, passes through preauricular points on both sides, curves posteriorly around the head to pass between the inion and the electrode positions O1, OZ, and O2 of the international 10-10 standard system, and then converges;
the coordinated irradiation condition of the irradiated surface area ratio and the delivered radiant power level comprises at least one of:
when the irradiated surface area ratio of the subject's head is from 30% to 40%, the spatiotemporally averaged irradiance over the irradiated surface of the subject's head is 117 mW/cm² or more and 230 mW/cm² or less;
when the irradiated surface area ratio of the subject's head is between 40% and 65%, the spatiotemporally averaged irradiance over the irradiated surface of the subject's head is 110 mW/cm² or more and 230 mW/cm² or less; or
when the irradiated surface area ratio of the subject's head is 65% or more, the average coordinated energy delivery to the subject's head ranges from 2750 W·% to 14100 W·%, wherein the average coordinated energy delivery is a product of the percentage value of the irradiated surface area ratio and the average total radiant power incident upon the subject's head, and the spatiotemporally averaged irradiance over the irradiated surface of the subject's head is 230 mW/cm² or less.

2. The phototherapy apparatus according to claim 1, wherein the coordinated irradiation condition of the irradiated surface area ratio and the delivered radiant power level indicates at least one of:
a correlation between the irradiated surface area ratio and the spatiotemporally averaged irradiance over the irradiated surface of the subject's head;
a product of the average total radiant power incident upon the subject's head and the percentage value of the total irradiated surface area ratio; or
an integral of the average radiant power incident upon each partition of the subject's head with respect to the irradiated surface area ratio of each partition.

3. The phototherapy apparatus according to claim 1, wherein the coordinated irradiation condition of the irradiated surface area ratio and the delivered radiant power level specifically comprises any one of:
when irradiation is delivered only to an anterosuperior cranial region of the subject's head, the spatiotemporally averaged irradiance over the irradiated surface of the subject's head is 117 mW/cm² or more and 230 mW/cm² or less;
when irradiation is delivered only to the anterosuperior cranial region and the cranial vertex region of the subject's head, or only the anterosuperior cranial region together with left lateral cranial region and right lateral cranial region of the subject's head, the spatiotemporally averaged irradiance over the irradiated surface of the subject's head is 110 mW/cm² or more and 230 mW/cm² or less;
when irradiation is delivered to the anterosuperior cranial region, the left lateral cranial region, the right lateral cranial region, and the cranial vertex region of the subject's head are irradiated, the average coordinated energy delivery to the subject's head ranges from 2750 W·% to 10800 W·%; or
when irradiation is delivered to the anterosuperior cranial region, the left lateral cranial region, the right lateral cranial region, the cranial vertex region, and posterior cranial region of the subject's head, the average coordinated energy delivery to the subject's head ranges from 2750 W·% to 14100 W·%.

4. The phototherapy apparatus according to claim 1, wherein the spatiotemporally averaged irradiance over the irradiated surface when the near-infrared light irradiation is delivered to a first proportion of the surface area of the reference calvarial region of the subject's head, is greater than the spatiotemporally averaged irradiance over the irradiated surface when the near-infrared light irradiation is delivered to a second proportion of the surface area of the reference calvarial region of the subject's head, wherein the first proportion is less than the second proportion.

5. The phototherapy apparatus according to claim 4, wherein the first proportion and the second proportion are selected from any one of the following:
both the first proportion and the second proportion are from 30% to 40%;
the first proportion is from 30% to 40% and the second proportion is between 40% and 65%;
both the first proportion and the second proportion are between 40% and 65%;
the first proportion is between 40% and 65% and the second proportion is 65% or more; or
both the first proportion and the second proportion are 65% or more.

6. The phototherapy apparatus according to claim 1, wherein the average total radiant power of the near-infrared light incident upon the subject's head is 23 to 140 W, or 29 to 120 W, or 31 to 100 W.

7. The phototherapy apparatus according to claim 1, wherein the subject's head comprises either the head of a patient or a reference head phantom of a population of treatment subjects, wherein the near-infrared light irradiation is delivered to any one of the following regions of the subject's head: the anterosuperior cranial region; the anterosuperior cranial region and the cranial vertex region; the anterosuperior cranial region, the left lateral cranial region and right lateral cranial region; the anterosuperior cranial region, the left lateral cranial region, the right lateral cranial region, and the cranial vertex region; and the anterosuperior cranial region, the left lateral cranial region, the right lateral cranial region, the cranial vertex region, and the posterior cranial region, and
the parameters of the reference head phantom of the population of treatment subjects are any one of the following sets of:
head width is 140 to 166 mm, head length is 170 to 196 mm, head circumference is 525 to 583 mm, morphological facial height is 104 to 130 mm, head sagittal arc is 304 to 372 mm, tragus-to-tragus arc is 320 to 375 mm, head height is 206 to 253 mm; or
head width is 152 mm, head length is 184 mm, head circumference is 536.7 mm, morphological facial height is 109.3 mm, head sagittal arc is 355.6 mm, tragus-to-tragus arc is 324.1 mm, head height is 206 mm.

8. The phototherapy apparatus according to claim 7, wherein according to the 10-10 standard system,
the anterosuperior cranial region forms a region including electrode positions FP2, FPZ, FP1, AF3, AF4, AF7, AF8, AFZ, FZ, F1, F2, F3, F4, F5, F6, F7, F8, FC1, FC2, FC3, FC4, and FCZ;
the cranial vertex region forms a region including electrode positions CZ, C1, C2, C3, C4, CPZ, CP1, CP2, CP3, CP4, PZ, P1, P2, P3, and P4;
the left lateral cranial region forms a region including electrode positions FT7, FC5, T7, C5, TP7, CP5, P7, and P5, the right lateral cranial region forms a region including electrode positions FT8, FC6, T8, C6, TP8, CP6, P8, and P6; and
the posterior cranial region forms a region including electrode positions PO7, PO5, PO3, POZ, PO4, PO6, PO8, O1, OZ, and O2.

9. The phototherapy apparatus according to claim 7,
wherein the anterosuperior cranial region is within a first region enclosed by a first boundary line and the total boundary line, according to the 10-10 standard system, the first boundary line sequentially passes between the following electrode positions: between F7 and FT7, between F5 and FC5, between FC3 and C3, between FC1 and C1, between FCZ and CZ, between FC2 and C2, between FC4 and C4, between F6 and FC6, and between F8 and FT8;
the cranial vertex region is within a second region enclosed by a second boundary line, according to the 10-10 standard system, the second boundary line sequentially passes between the following electrode positions: between FC3 and C3, between FC1 and C1, between FCZ and CZ, between FC2 and C2, between FC4 and C4, between C6 and C4, between CP6 and CP4, between P6 and P4, between PO4 and P4, between PO4 and P2, between POZ and P2, between POZ and PZ, between POZ and P1, between PO3 and P1, between PO3 and P3, between P5 and P3, between CP5 and CP3, and between C5 and C3;
the left lateral cranial region is within a third region enclosed by a third boundary line and the total boundary line, according to the 10-10 standard system, the third boundary line sequentially passes between the following electrode positions: between FT7 and F7, between FC5 and F5, between FC5 and FC3, between C5 and C3, between CP5 and CP3, between P5 and P3, between P5 and PO5, and between P7 and PO7;
the right lateral cranial region is within a fourth region enclosed by a fourth boundary line and the total boundary line, according to the 10-10 standard system, the fourth boundary line sequentially passes between the following electrode positions: between FT8 and F8, between FC6 and F6, between FC6 and FC4, between C6 and C4, between CP6 and CP4, between P6 and P4, between P6 and PO6, and between P8 and PO8;
the posterior cranial region is within a fifth region enclosed by a fifth boundary line and the total boundary line, according to the 10-10 standard system, the fifth boundary line sequentially passes between the following electrode positions: between P7 and PO7, between P5 and PO5, between P3 and PO3, between P1 and POZ, between PZ and POZ, between P2 and POZ, between P4 and PO4, between P6 and PO6, and between P8 and PO8.

10. The phototherapy apparatus according to claim 9, wherein the surface area of the anterosuperior cranial region constitutes at least 70% of the surface area of the first region, the surface area of the cranial vertex region constitutes at least 70% of the surface area of the second region, the surface area of the left lateral cranial region constitutes at least 70% of the surface area of the third region, the surface area of the right lateral cranial region constitutes at least 70% of the surface area of the fourth region, and the surface area of the posterior cranial region constitutes at least 70% of the surface area of the fifth region.

11. The phototherapy apparatus according to claim 7, wherein when the irradiation is delivered to the anterosuperior cranial region, the left lateral cranial region, the right lateral cranial region, and the cranial vertex region, or when the irradiation is delivered to the anterosuperior cranial region, the left lateral cranial region, the right lateral cranial region, the cranial vertex region, and the posterior cranial region,
the spatiotemporally averaged irradiance over the irradiated surface of the anterosuperior cranial region is 50 to 120 mW/cm², the spatiotemporally averaged irradiance over the irradiated surface of the cranial vertex region is 60 to 120 mW/cm², and the spatiotemporally averaged irradiance over the irradiated surface of the left lateral cranial region and right lateral cranial region is 32 to 85 mW/cm².

12. The phototherapy apparatus according to claim 7, wherein when the irradiation is delivered to at least the anterosuperior cranial region, the left lateral cranial region and the right lateral cranial region, and cranial vertex region,
the time-averaged irradiance at the electrode positions Fp1, Fpz, Fp2, AF3, AFz, AF4, F5, F3, F1, Fz, F2, F4, F6, FC1, FC2, FC3, FC4, FC5, FC6, C1, C2, C3, Cz, C4, C5, C6, CP1, CP2, CPz, CP3, CP4, P1, P2, and Pz is 50-90 mW/cm², and
the time-averaged irradiance at the electrode positions FCz, TP7, FT7, T7, FT8, T8, POZ, P6, TP8, CP6, P5, CP5, F7, F8, AF7, and AF8 is 25-65 mW/cm².

13. The phototherapy apparatus according to claim 1, wherein the unit energy delivery to the subject's head is the irradiation of at least 8260 to 42250 Joules delivered to the subject's head during a continuous irradiation period of 5 minutes.

14. The phototherapy apparatus according to claim 1, wherein a daily cumulative energy delivery to the subject's head is the irradiation of at least 24800 to 1014050 Joules delivered to the subject's head within a single day in separate single-session cumulative energy deliveries, when if the single-session cumulative energy delivery is performed by using a cumulative irradiation time of at least 30 minutes within a single session of one hour, the single-session cumulative energy delivery can be administered up to four times within a single day to provide an accelerated daily cumulative energy delivery.

15. The phototherapy apparatus according to claim 1, wherein adjacent near-infrared irradiation units have a predetermined spacing therebetween and each near-infrared irradiation unit has a predetermined emission angle, such that, when the subject's head is positioned in place within the accommodation space, near-infrared light is emergent from each position on a peripheral irradiation curved surface corresponding to a reference calvarial region of the subject's head.

16. The phototherapy apparatus according to claim 1, wherein the array of near-infrared irradiation units are specifically configured to emit near-infrared light with a duty cycle of 30% to 70%, a wavelength of 650 to 1100 nm, and a frequency falling within the Alpha wave frequency range, the Gamma wave frequency range, or a frequency range adjacent thereto, wherein the time-averaged radiant power of each individual near-infrared irradiation device constituting the near-infrared irradiation unit is 90 mW or more.
